SEQUENCE LISTING (16 sequences)

SEQ ID NO: 1 - DNA, 1483 bp, Zea mays
  CDS (121)..(1155)
ccacgcgtccggcaaatacaagctctcagttagaccacactcaaaccagccacggacagcctcaaaatcccgagcaggagctcgctcgtagccccgagaacccgaaagctctgcagcgagatgtctcccgagctgaccggcaagatggccgccaaggccgccgccgccgctgcggcggtgaagcccgcgacgagggcgtacgtgacgttcctggcgggcaacggcgactactggaagggcgtggtgggcctggccaagggcctgcgcaaggtcggctcggcctatcccctggtggtggcccttctgcccgacgtgccggagtcccaccgccgcatcctcgtctcgcagggctgcatcctccgcgagatcgagcccgtgtacccgcctgagaaccagacgcagttcgccatggcgtactacgtcatcaactactccaagctccgcatctgggagttcgtggagtacgagaagatggtgtacctggacgcggacatccaggtgttcgagaacatcgacgagctgttcgagctcgagaaagggtacttctacgcggtgatggactgcttctgcgaaaagacgtggagccacaccccgcagtacaagatcgggtactgccaacagtgcccggacaaggtgacgtggccgaccaccgagctgggccctccgcccccgctctacttcaatgccgggatgttcgcgcacgagcccagcatggccaccgccaaggcactcctcgacaccctccgcgtcactccgcccacccccttcgcagagcaagacttcctgaacatgttcttcagggaccagtacaggccgatccctaacgtgtacaacctcgtgctccccatgctctggaggcaccccgagaacgtgcaacttgagaaagtgaaagtcgtgcactactgcgcggcggggtccaagccgtggaggttcacgggcaaggaggccaacatggacagggaggacatcaagtcgctggtgaacaagtggtgggacatctacaacgacgagtcgctggacttcaagggcctgcccctctcgcccgccgacgccgacgccgacgacgaggtggaggcggtggccaagaagccgctccgcgcggcgctggcggaggccggcacggtcaagtacgtcaccgcgccctcggccgcgtgaccccgtgcttcggcttcccgccggtggccgcgctatggcttttgcgtagtcgtcacaagttcgtaaagaagaatctcactatatggcctttgttttgaattttgtttatgaattagggtgtctaattttggatcagttcctttcccttgtgtgtctaattagggtgtgagcttatatatataagtaccaattcaaggtgcaagcgttcgtcaggaattgggctttatggatacgcccttcggtcacagctagagtatttgtcctgtaccgagggattatgtttattttgcttggtgaattaatatataaaatacgaagctgcgaaaat

SEQ ID NO: 2 - protein, 344 aa, Zea mays
MSPELTGKMAAKAAAAAAAVKPATRAYVTFLAGNGDYWKGVVGLAKGLRKVGSAYPLVVALLPDVPESHRRILVSQGCILREIEPVYPPENQTQFAMAYYVINYSKLRIWEFVEYEKMVYLDADIQVFENIDELFELEKGYFYAVMDCFCEKTWSHTPQYKIGYCQQCPDKVTWPTTELGPPPPLYFNAGMFAHEPSMATAKALLDTLRVTPPTPFAEQDFLNMFFRDQYRPIPNVYNLVLPMLWRHPENVQLEKVKVVHYCAAGSKPWRFTGKEANMDREDIKSLVNKWWDIYNDESLDFKGLPLSPADADADDEVEAVAKKPLRAALAEAGTVKYVTAPSAA

SEQ ID NO: 3 - DNA, 1481 bp, Zea mays
  CDS (111)..(1157)
ccacgcgtccgagcgcttaaaccacactcaaaccaaccgccagccaacaaacaggcctcctagtcccgaccaagaactcgctcgtagccccgagaacccgacagcgcgagatggctcccgagctgatgacagccaagatgaccgccaatgccgccgcggcggcggcggcggcggcggcggcgaagcctgccacgagggcgtacgtgacgttcctggcgggcgacggcgactactggaagggcgtggtggggctggccaagggcctccgcaaggtccgctcggcctaccccctggtggtggccgtgctgcccgacgtgcccgagtcccaccgccgcatcctcgtctcgcagggctgcgtcgtccgcgagatcgagcccgtgtacccgcctgagaaccagacgcagttcgccatggcgtactacgtcatcaactactccaagctccgcatctgggagttcgtggagtacgagaggatggtgtacctggacgcggacatccaggtgttcgagaacatcgacggcctgttcgagctcgagaaagggtacttctacgcggtgatggccggcttctgcgaaaaaacgtggagccacaccccgcagtacaggatcggctactgccaacagtgcccggacaaggtggcgtggccaacgcgaacggccgagctgggcctcccgccgtcctcctacttcaacgccggaatgttcttgaaggagccaaacttggcaaccgcaaaggggtcccccgaaaccctccggttgattccgccaacccccttcgcaaagaaggacttcctgaaaatgttcttcagggataattacaggccgatccccaacgtttacaacctcgtgctggccatgctttggaggcaccccgagaacgtgcagctggagaaggtgaaggtcgtgcattattgcgcggcggggtccaagccgtggaggttcacgggcaaggaggcgaacatggacagggaggacatcaacgccctggtgaagaagtggtgggacatctacaacgacgagacgctggacttgaagggctgcctcgcttctcgcccgacgacgacgacgaggtggaggcggtgcccaagaagccgcttcgcgcggccttggcggaggccggcacggtcaaagtacgtcaccgcgccctcggccgcgtgatcccgtgctctggccttccccaccgcgcaccggtggccgcgcgctatagcttcgtgtgtggccatcgcggaacataagaagaatctctcactataaagggccttctgtgtttgcattttggttttggattagggtgtctaatttttcaccagttccttttccccttgtgtaagtgtggagcttactatatagctagcgcccatttcaaggtgctgcaagcgttggtcaggaattgggctagagtgtttgtcctgtaccgagggattatgtgtatttttgctatttcgctcggtataattcatccattaataaatagggagccgc

SEQ ID NO: 4 - protein, 348 aa, Zea mays
MAPELMTAKMTANAAAAAAAAAAAKPATRAYVTFLAGDGDYWKGVVGLAKGLRKVRSAYPLVVAVLPDVPESHRRILVSQGCVVREIEPVYPPENQTQFAMAYYVINYSKLRIWEFVEYERMVYLDADIQVFENIDGLFELEKGYFYAVMAGFCEKTWSHTPQYRIGYCQQCPDKVAWPTRTAELGLPPSSYFNAGMFLKEPNLATAKGSPETLRLIPPTPFAKKDFLKMFFRDNYRPIPNVYNLVLAMLWRHPENVQLEKVKVVHYCAAGSKPWRFTGKEANMDREDINALVKKWWDIYNDETLDLKGCLASRPTTTTRWRRCPRSRFARPWRRPARSKYVTAPSAA

SEQ ID NO: 5 - DNA, 1487 bp, Zea mays
  CDS (129)..(1166)
ccacgcgtccgctcaagcaaaaacacaaagcgcttaaaccacactcaaaccaaccgccaacaaacaggcctcctagtcccgaccagaacttgctcgtagccccgcccccgagaacccgacagcgcgagatggctcccgagctgatgacagccaagatgaccgccaaagccgcggcggcggcggcggcggtgaagcctgccacgagggcgtacgtgacgttccttgcgggcgacggcgactactggaagggcgtggtggggctggccaaaggcctgcgcaaggtccgctcggcctaccccctggtggtggcggtgctgcccgacgtgcccgagtcccaccgccgcatcctcgtctcgcagggctgcgtcgtccgcgagatcgagcccgtgtacccgcctgagaaccagacgcagttcgccatggcgtactacgtcatcaactactccaagctccgcatctgggagttcgtggagtacgagaggatggtgtacctggacgcggacatccaggtgttcgagaacatcgacggcctgttcgagctcgagaaggggtacttctacgcggtgatggactgcttctgcgagaagacgtggagccacaccccgcagtacaggatcggctactgccagcagtgcccggacaaggtggcgtggccagcggcgaccgccgagctgggccctccgccgtcgctctacttcaacgccggcatgttcgtgcacgagcccagcgtggccaccgccaaggcgctcctcgacaccctccgcgtgactccgcccacccccttcgcagagcaggacttcctgaacatgttcttcagggatcagtacaggccgatccccaacgtgtacaacctcgtgctggccatgctctggaggcaccccgagaacgtgcagctggagaaggtgaaggtcgtgcactactgcgcggcggggtccaagccgtggaggttcacgggcaaggaggcgaacatggacagggaggacatcaacgccctggtgaacaagtggtgggacatctacaacgacgagacgctggacttgaagggcctgccctcgctctcgcccgacgacgacgacgaggtggaggcggtggccaagaagccgcttcgcgcggccctggcggaggccggcacggtcaagtacgtcaccgcgccctcggccgcgtgatcccgtgctctggccttcccccaccgcgcaccgtaccggtggccgcgcgctatagcttgtgtgtgtgtggccatcgcggaacataagaagaatctctcactataaaggccttcgttctgtgtttgcattttggttttggattagggtgtctaatttttcaccagttccttttccccttgtgtaagtgtggagcttactatatagctagcgcccatttcaaggtgctgcaagcgttggtcaggaattgggctagagtgtttgtcctgtaccgagggattatgtgtatttttgctatttcgctcggtaaattaatacaatatt

SEQ ID NO: 6 - protein, 345 aa, Zea mays
MAPELMTAKMTAKAAAAAAAVKPATRAYVTFLAGDGDYWKGVVGLAKGLRKVRSAYPLVVAVLPDVPESHRRILVSQGCVVREIEPVYPPENQTQFAMAYYVINYSKLRIWEFVEYERMVYLDADIQVFENIDGLFELEKGYFYAVMDCFCEKTWSHTPQYRIGYCQQCPDKVAWPAATAELGPPPSLYFNAGMFVHEPSVATAKALLDTLRVTPPTPFAEQDFLNMFFRDQYRPIPNVYNLVLAMLWRHPENVQLEKVKVVHYCAAGSKPWRFTGKEANMDREDINALVNKWWDIYNDETLDLKGLPSLSPDDDDEVEAVAKKPLRAALAEAGTVKYVTAPSAA

SEQ ID NO: 7 - DNA, 2278 bp, Zea mays
  CDS (200)..(2278)
cacgcgtccgcaggtagtagcttagctagcgtgataatcaccgcgctaaactgatcatatatacacagcaattgtcgtgtgacaggacagaggaggaggggcaggctggtaatatctagcaaaagtacaagcgttcctccagtggcgtccagcgctatatagttcgctgtccggcgtcctttccgaccgagcgcgagcgatgacggtgacgccgaggattacggtgggggatgggcggctggtggcgcacgggcggagcgtcctcgccggcgtgccggacaacatcgtgctcacgcacgcctccggggcggggctggtcgacggcgccttcgtcggcgccacggccgcggagcccagcagcatgcacgtcttcaccttcggcaccctccgggacctgcgcttcgtgtgctgcttccggttcaagctgtggtggatgacccagcgcatgggcacctccggccgcgacgtcccgctggagacgcagttcatgctcctggagtgcggcccgggcgacggcgacgcggcggcgtacgtggcgatgctgccgctgctggagggccagttccgggcggcgctgcagggcaacgaccgcgacgagctggagatcaccttcgagagcggggacaaggcggtgcagactgcgcatgggacctgcatgctgtacgtgcacgccgggaccaaccctttcgacaccatcactcaggcggtcaaggtggtggagagacacatgcagacgttccatcacagggagaagaaaaagctgccgtcgttcgtggactggttcggctggtgcacgtgggacgccttctacaccgacgtcaccgccgagagcgtcaagcaaggcctcaagagcttggcggacggcggcacgccgccgcggttcctgatcatcgacgacgggtggcagcagatcggcagcgagaacaaggaggagtccgccaacgccgtcgtccaggaagggcagttcgccagtaggctgacggggatcaaggagaacgccaagttccagaagaagacgaagacgaaggagagcggcggcggcggcggcgagcagcaggcgcaaacgccgggcctgaagctgctggtggaggaggcgaagcgcgagcacggcgtcaggtacgtgtacgtgtggcacgccatggcggggtactggggcggcgtgaagccggcggcggaggggatggagcactacgagagcgcgctggcgtacccggtgcagtcgccgggtgtgatgggcaaccagccggacatcgtcatggactctctgtccgtgctgggcctggggctggtgcacccgcgcagggcgctgagcttctacgacgagctgcactcgtacctcgcctcctgcggcgtcgatggcgtcaaggtggacgtgcagaacatcatcgagacgctcggcgccgggcacggcggccgggtcgcgctcacccgcgcttaccaccgcgcgctcgaggactccgtggcgcgcaacttccccgacaacggctgcatctcctgcatgtgccacaacaccgacatgctctacagcgcgcgccagaccgccgtcgtgcgcgcctccgacgacttctacccgcgcgacccggcgtcgcacaccgtccacgtctcctccgtcgcctacaacaccctcttcctcggcgagttcatgcagcccgactgggacatgttccatagcctgcacccggcggcggactaccacggcgcggcgagggcaatcggcggctgcccgatatacgtcagcgacaagccaggcaaccacaacttcgagctgctcaagaagctggtgctccccgacggctccgtgctccgcgcgcagctcccgggccgccccacccgcgattgcctcttcgccgacccggcgcgcgacggcacgagcctgctcaagatctggaacgtgaacaagtgcacgggcgtggtgggcgtgttcaactgccagggcgccgggtggtgccgcgtcaccaagaagacccgggtgcacgacgcggcgccggggacgctgacggggtcggtccgcgccgacgacgtggacgtgatcgccggcctcgccggggacggctggggcggcgaggccgtcgtgtacgcctacaggtcaggggagctagtcaggctgccccagggcgccacactgccggtgacgctcaaggtgctggagttcgaggtgttccacgtctcccccgtcagggcggtcgcgccgggcgtgtcgttcgcgcccatcggcctgctcgacatgttcaactccggcggggcggtggagcagtgcgaggtccgtggcggcgcgaac

SEQ ID NO: 8 - protein, 693 aa, Zea mays
MTVTPRITVGDGRLVAHGRSVLAGVPDNIVLTHASGAGLVDGAFVGATAAEPSSMHVFTFGTLRDLRFVCCFRFKLWWMTQRMGTSGRDVPLETQFMLLECGPGDGDAAAYVAMLPLLEGQFRAALQGNDRDELEITFESGDKAVQTAHGTCMLYVHAGTNPFDTITQAVKVVERHMQTFHHREKKKLPSFVDWFGWCTWDAFYTDVTAESVKQGLKSLADGGTPPRFLIIDDGWQQIGSENKEESANAVVQEGQFASRLTGIKENAKFQKKTKTKESGGGGGEQQAQTPGLKLLVEEAKREHGVRYVYVWHAMAGYWGGVKPAAEGMEHYESALAYPVQSPGVMGNQPDIVMDSLSVLGLGLVHPRRALSFYDELHSYLASCGVDGVKVDVQNIIETLGAGHGGRVALTRAYHRALEDSVARNFPDNGCISCMCHNTDMLYSARQTAVVRASDDFYPRDPASHTVHVSSVAYNTLFLGEFMQPDWDMFHSLHPAADYHGAARAIGGCPIYVSDKPGNHNFELLKKLVLPDGSVLRAQLPGRPTRDCLFADPARDGTSLLKIWNVNKCTGVVGVFNCQGAGWCRVTKKTRVHDAAPGTLTGSVRADDVDVIAGLAGDGWGGEAVVYAYRSGELVRLPQGATLPVTLKVLEFEVFHVSPVRAVAPGVSFAPIGLLDMFNSGGAVEQCEVRGGAN

SEQ ID NO: 9 - DNA, 1590 bp, Zea mays
  CDS (435)..(1589)
ccacgcgtccgggctcacggtcacggcctcacccatccgcttgccatccaatccgacccggccccatcgagaacgtgcgcgtgcgcgctgcctaggctgatacgagcagccagcaagcaagcaacagatttaaccgccgccgatgtccgtgatggcgcggaggctgagctccagctgcagcagggccgtcgcgtcctcgtgcccttccaaatcttctctgcggcgccgtatatatacccttccttcccgcccgctgctactcgcattctcgctgcgggcctcgcctctgtgtccgggcgccgcctctccctcctcccaccccgcctcggtgttcgtttaggactctcgcggcggccaaggagtcgtcgtcgccgccgtcgaccagcgtctcgccgacccagcagcagtcccggagtacgatagagggaggcgagatgacggtcgcctcgtccgtcaggctcgccggcggcaatctgacggtatgcgggcggacggtgctgtccggggtgccggacgcggtggtggccacgtcggcggcgacggagggagcggtcgacgggatcttcctcggcgccgacttcgccgagccggccgcccggcacgtcgtctccctcggcgacctgagggacgttcggttcatggcgtgcttccgattcaagctatggtggatggcgcagcggatgggggagaaaggcagcgacgtcccgcgcgagacccagttcctgctcgtcgagtccaggggcgtcggcgacgaggacgcggcgtacgtcgtgttcctcccgctcgtggagggcgcgttccgggccagcatccaaggcggtgccggcgacgcgctggagctctgcgtcgagagcggggacgacgacacgcgcgcggcgtccttcgagcgatccctcttcgtgggcgccgcggagtccgaccccttcgcggccatctccggcgcagtcgccgccgccaagtccgcgctcaggacgttccgggtccgcgccgagaagaagctcccgggcatcgtcgactacttcggctggtgcacctgggacgccttttaccaggacgtcacccaggagggcgtcgaggccgggctccgcagcctcatcgccggcggcgcgccgcccaagttcgtcatcatcgacgacggctggcagtccgtcgccaccgacaccaacgaatcggccggagaggacaagccgcccctcctatctcggctcaccggcatcaaggagaacagcaagttccagaacgcggacgacccggccgccggcatcaagacggtggtgcgcttggcgaaggaggagtaccggctcaagtacgtctacgtgtggcacgccatcaccggctactggggcggcgtccggcccggcgaggagcactaccgctccagcatgcagttccccaaggtttcgccgggcgtcatggagaacgagcccggcatgaagaccgacgtgctcaccgtgcaggggctcggcctcgtgcacccgcgcgccgtgtaccgcttctacgacgagctccacgcgtacctcgccgccgccggagtcgacggcgtcaaggtggacgtgcagtgcatcctggagacgctcggcgccggccacg

SEQ ID NO: 10 - protein, 385 aa, Zea mays
MTVASSVRLAGGNLTVCGRTVLSGVPDAVVATSAATEGAVDGIFLGADFAEPAARHVVSLGDLRDVRFMACFRFKLWWMAQRMGEKGSDVPRETQFLLVESRGVGDEDAAYVVFLPLVEGAFRASIQGGAGDALELCVESGDDDTRAASFERSLFVGAAESDPFAAISGAVAAAKSALRTFRVRAEKKLPGIVDYFGWCTWDAFYQDVTQEGVEAGLRSLIAGGAPPKFVIIDDGWQSVATDTNESAGEDKPPLLSRLTGIKENSKFQNADDPAAGIKTVVRLAKEEYRLKYVYVWHAITGYWGGVRPGEEHYRSSMQFPKVSPGVMENEPGMKTDVLTVQGLGLVHPRAVYRFYDELHAYLAAAGVDGVKVDVQCILETLGAGH

SEQ ID NO: 11 - DNA, 2483 bp, Zea mays
  CDS (104)..(2347)
ccacgcgtccgcgccagccaactatcgccgctgataaccaacccaatccaacctaacccaaccctgctctctgccgacgcggattcgaatccgaggacgatcgatgacggtgggcgccggcatcgccgtccaggacggcagcctcctggcgctgggcgccaaggtcctgcgggaggtgcgcggcaacgtgctcgtgacgccggccgccggcgggggcctaaccaacggcgcgttcctcggcgtccggtccgcgcccgccgccagccgcagcatcttccccgtcgggaagctccgggaccagcggttcgtgtgcacgttccggttcaagatgtggtggatgacgcaaaggatgggctccgcaagccgcgacatcccctccgagacgcaatttctgctggtggaaaggtccggcggcggcgaacaacccgtcgtgtacaccgtcttcctccccgtgctggagggctcgttccgtgccgttcttcaggggaacgccgccgacgagctggagatctgcctggagagcggcgacccggacgtggaatctttccaaggctcccatctggtcttcgttggcgccggatcggacccgttcgaggtcatcacaagttcagtcaaggctgtcgagaggcacttgcagacgttctctcacagggagaagaaaaagatgccagacattctgaactggtttggctggtgcacgtgggacgcgttctacaccaatgtcaacgcccagggagtgaagcaaggattgcagagcttggaaaaaggcggggtctctcctaggttcgtcataatcgacgacggatggcagtccgtcgccatggaccctgtgggaatcgcttgcctatctgacaactcagccaacttcgcaaacaggctgactcacatcagggagaaccacaagtttcagaaaaatggcagggagggtcacagggaagatgacccagcgaagggcctagcacacgtcgtcaatgagattaaggggaagcatcagctcaagtatgtgtacgtatggcatgccatcaccggatactggggcggagtgaggccgggtgcagctggaatggagcactacggatcaaagatgcagcggcccgtgccatcgccgggggttcagaagaacgagcgctgcgacgccctggacagcatgacggccaacgggctgggcctcgtgaaccctgacagggcgttcagtttctacgacgagctccactcgtacctcgcgtctgccgggatcgacggggtgaaggtggacgtgcagaacgtcctcgagacgctgggcgccggccatggcgggagggtgatgctggcgaggaagtaccagcaggctctggaagcgtccgtcgccaggaacttccctgacaatggcatcatatcgtgcatgagccacagcacggacaacttgtacagctcgaaacggagcgcggtgattagagcctctgacgatttctggccgagagaccccgcttcccataccatacacgtcgcgtccgtcgcttataacaccgtctttcttggggagttcatgcagccagactgggacatgttccatagtgttcatcccatggctgagtaccatgctgcggctcgagcggttggtggctgtgccatatatgtcagcgacaagcctgggagccatgacttcaatctgctcaagaagctcgtgcttcccgacggatcgatcctgcgcgcaaagctccccgggaggccaaccagagactgcctcttctctgaccccgcaagggacggcaaaagcgttctcaagatatggaacttgaacgaacactccggcgtcgttggcgccttcaactgccaaggcgccggctggtgccgggtagccaagaagaacctcatccacgaccagcagcccggaacgttgagcggcgtcatccgggcgcaggacgtggagcaccttggaagggtggctgatcacggttggaacggcgacgtggtcgtgtatttgcacgtgggaggggaggtggtgtacctgccgaagaacgccttgctgcctgtgacgctgagatcgcgcgagtatgaggtgttcaccgtcgtccctctcaagcacctgccaaacggtacctcctttgcggcgatcggccttctcggcatgttcaactccggtggcgcggtgagggagctgagattcggtggtgaggatgccgacgtcgagctcagagtgcggggctcgggcacggtcggagcttattcctcgaccaagccaacgtgtgtcgccgtcgattccaaggcggttggtttctcctacgatgccacctgtggcctcatcagcttcgagctcggcattcccgaccaagaaatgtacttgtggacggttacagtagagtattgaagttacaactgagaattgagatagattttctaatgcgtcagagaacgcccggtggtgtaggtattctgatttattcagtgttgtactttctattgttagttatatatattctgtacacatcccgggattttatgac

SEQ ID NO: 12 - protein, 747 aa, Zea mays
MTVGAGIAVQDGSLLALGAKVLREVRGNVLVTPAAGGGLTNGAFLGVRSAPAASRSIFPVGKLRDQRFVCTFRFKMWWMTQRMGSASRDIPSETQFLLVERSGGGEQPVVYTVFLPVLEGSFRAVLQGNAADELEICLESGDPDVESFQGSHLVFVGAGSDPFEVITSSVKAVERHLQTFSHREKKKMPDILNWFGWCTWDAFYTNVNAQGVKQGLQSLEKGGVSPRFVIIDDGWQSVAMDPVGIACLSDNSANFANRLTHIRENHKFQKNGREGHREDDPAKGLAHVVNEIKGKHQLKYVYVWHAITGYWGGVRPGAAGMEHYGSKMQRPVPSPGVQKNERCDALDSMTANGLGLVNPDRAFSFYDELHSYLASAGIDGVKVDVQNVLETLGAGHGGRVMLARKYQQALEASVARNFPDNGIISCMSHSTDNLYSSKRSAVIRASDDFWPRDPASHTIHVASVAYNTVFLGEFMQPDWDMFHSVHPMAEYHAAARAVGGCAIYVSDKPGSHDFNLLKKLVLPDGSILRAKLPGRPTRDCLFSDPARDGKSVLKIWNLNEHSGVVGAFNCQGAGWCRVAKKNLIHDQQPGTLSGVIRAQDVEHLGRVADHGWNGDVVVYLHVGGEVVYLPKNALLPVTLRSREYEVFTVVPLKHLPNGTSFAAIGLLGMFNSGGAVRELRFGGEDADVELRVRGSGTVGAYSSTKPTCVAVDSKAVGFSYDATCGLISFELGIPDQEMYLWTVTVEY

SEQ ID NO: 13 - DNA, 20 bp, Zea mays
gaccacactcaaaccagcca

SEQ ID NO: 14 - DNA, 35 bp, Zea mays
taatacgactcactatagggcagaagggccaccac

SEQ ID NO: 15 - DNA, 20 bp, Zea mays
aaccgccagccaacaaacag

SEQ ID NO: 16 - DNA, 34 bp, Zea mays
taatacgactcactatagggtcgcccgccaggaa

(19) United States
(12) Patent Application Publication
(10) Pub. No.: US 2005/0055748 A1
(43) Pub. Date: Mar. 10, 2005

(54) Stress-responsive genes, regulatory elements, and methods of use for same

(76) Inventors: A. Bruce Downie, Lexington, KY (US); Tian-Yong Zhao, Lexington, KY (US); Robert Meeley, Johnston, IA (US)

Correspondence Address: MCKEE, VOORHEES & SEASE, P.L.C.;ATTN: PIONEER HI-BRED, 801 GRAND AVENUE, SUITE 3200, DES MOINES, IA, 50309-2721, US

(21) Appl. No.: 10/812,271

(22) Filed: Mar. 29, 2004

Related U.S. Application Data
(60) Provisional application No. 60/458,339, filed on Mar. 28, 2003.

(51) Int. Cl. A01H 1/00; C12N 15/82; C07H 21/04; A01H 5/00

(52) U.S. Cl. 800/289; 800/312; 435/468; 435/415; 536/23.6

(57) ABSTRACT

The present invention relates to stress-regulated polypeptides, nucleotide sequences encoding the same and regulatory elements and their use in creating or enhancing stress tolerance in plants. Nucleic acid constructs comprising a stress-responsive sequence involved in the production of raffinose operably linked to a promoter, or other nucleotide sequence operably linked to a stress specific regulatory region are disclosed as well as vectors, plant cells, plants, and transformed seeds containing such constructs are provided. Methods for the use of such constructs in repressing or inducing expression of a stress-control sequences in a plant are also provided. In addition, methods are provided for conferring or improving stress tolerance in plants by repression or induction of stress-regulatory sequences or by spatially and temporally directing expression to periods of stress.

### CROSS-REFERENCE TO RELATED APPLICATIONS

This application claims priority to Provisional Application Ser. No. 60/458,339, filed Mar. 28, 2003, herein incorporated by reference in its entirety.

### GRANT REFERENCE

This work was supported in part by the U.S. Department of Agriculture National Research Initiative Competitive Grants Program (Grant No. 98-35100-6082) and Hatch funds. The government may have certain rights in this invention.

### FIELD OF THE INVENTION

This invention relates to novel compositions and methods useful in creating or enhancing stress-resistance in plants. Additionally, the invention relates to plants and other organisms which have been genetically transformed with the compositions of the invention.

### BACKGROUND OF THE INVENTION

Abiotic environmental stresses, such as drought stress, salinity stress, heat stress, and cold stress, are major limiting factors of plant growth and productivity. Crop losses and crop yield losses of major crops such as rice, maize (corn) and wheat caused by these stresses represent a significant economic and political factor and contribute to food shortages in many underdeveloped countries.

Drought, cold as well as salt stresses have a common theme important for plant growth and that is water availability. Most plants have evolved strategies-to protect themselves against these conditions of desiccation. However, if the severity and duration of the drought conditions are too great, the effects on plant development, growth and yield of most crop plants are profound. Since high salt content in some soils result in less available water for cell intake, its effect is similar to those observed under drought conditions. Continuous exposure to drought and high salt causes major alterations in the plant metabolism. These great changes in metabolism ultimately lead to cell death and consequently yield losses. Additionally, under freezing temperatures, plant cells lose water as a result of ice formation that starts in the apoplast and withdraws water from the symplast.

Developing stress-tolerant plants is a strategy that has the potential to solve or mediate at least some of these problems. However, traditional plant breeding strategies to develop new lines of plants that exhibit resistance (tolerance) to these types of stresses are relatively slow and require specific resistant lines for crossing with the desired line. Limited germplasm resources for stress tolerance and incompatibility in crosses between distantly related plant species represent significant problems encountered in conventional breeding. Additionally, the cellular processes leading to drought, cold and salt tolerance in model, drought- and/or salt-tolerant plants are complex in nature and involve multiple mechanisms of cellular adaptation and numerous metabolic pathways. This multi-component nature of stress tolerance has not only made breeding for tolerance largely unsuccessful, but has also limited the ability to genetically engineer stress tolerance plants using biotechnological methods.

Thus, it would be desirable to utilize recombinant DNA technology to produce new plant varieties and cultivars in a controlled and predictable manner. It would be especially desirable to produce crop and ornamental plants with improved stress tolerance over a range of environmental conditions to increase yield potential.

Consequently, there is a continued need for the identification of stress responsive genes to understand and improve a plant's response and resistance to stress to improve health and yield.

It is an object of the present invention to provide novel methods and compositions for improving stress tolerance in plants.

It is a further object of the invention to identify and characterize genes involved in the metabolic pathways that result in a plants response to stress.

It is yet another object of the invention to use genetic engineering techniques to provide nucleic acids, vectors, proteins and transgenic plants with a manipulated response to stress to improve health and yield of such plants.

### SUMMARY OF THE INVENTION

Compositions and methods involved in the stress response pathways for promoting stress tolerance in plants are provided. The compositions include nucleic acid molecules comprising a sequence useful in stress response as well as stress induced regulatory elements. The invention further includes expression constructs comprising nucleic acid sequences, operably linked to regulatory promoter elements, including the stress regulatory promoter elements of the invention, the nucleic acid sequences encoding proteins useful in stress tolerance of the invention or other combinations of these novel sequences of the invention with other nucleotide sequences, as well as vectors and transformed plant cells, plants and seeds comprising these constructs. The stress tolerance sequences include novel proteins which are up regulated in response to stress and lead to the production of raffinose, a galactosyl-sucrose trisaccharide that accumulates in maize during seed development. These proteins, the nucleotide sequences encoding them and the regulatory elements associated with them provide an opportunity to manipulate stress response pathways in plants to engineer plants to provide improved resistance to drought, salinity, temperature, and other stressors. The proteins of the invention include galactinol synthase (GOLS) and raffinose synthase (RAFS) which work in concert to produce raffinose. Amino acid sequences of these proteins are provided. Polynucleotides having nucleic acid sequences encoding maize GOLS and RAFS also provided. The DNA sequences encoding these proteins can be used to transform plants, bacteria, fungi, yeasts, and other organisms for improved stress tolerance.

In yet another embodiment regulatory regions capable of conferring spatial and temporal expression that is stress specific are provided. These comprise regulatory elements such as promoters that are natively associated with the nucleotide sequences encoding the proteins of the invention as well as their functional equivalents. These promoters provide a unique expression profile that is stress induced and particularly directed to seed development, a critical time in plant development when the plant is most vulnerable to environmental stress. In addition to these promoter sequences, the promoters of the invention encompass fragments and variants of these particular promoters as defined herein. Further the nucleotide sequences encoding the proteins disclosed herein can be used to isolate promoters of the genes of the invention using standard molecular protocols as described and incorporated by reference herein. These promoter elements can also be used to isolate other signaling components associated with regulation of these genes in response to drought, salinity, temperature mineral stress or other abiotic stresses.

The polynucleotides of the invention of at least 20 contiguous bases therefrom may be used as probes to isolate and identify similar genes in other plant species.

In one aspect, this invention relates to DNA sequences isolated from maize (Zea mays). These sequences alone, or in combination with other sequences, can be used to improve the stress tolerance of a plant. In another aspect of the present invention, expression cassettes and transformation vectors comprising the isolated nucleotide sequences are disclosed. The transformation vectors can be used to transform plants and express the stress control genes in the transformed cells. In this manner, the stress tolerance, of plants can be improved. Transformed cells as well as regenerated transgenic plants and seeds containing and expressing the isolated DNA sequences and protein products are also provided.

### BRIEF DESCRIPTION OF THE DRAWINGS

FIG. 1 is a phylogenetic evaluation of GOLS and RAFS. All known, full-length protein sequences for; A) GALACTINOL SYNTHASE or; B) RAFFINOSE SYNTHASE were subjected to clustal analysis and the resulting alignments used to construct optimized phylogenetic trees based on a heuristic search using random stepwise addition of taxa and replicated 100 times. Five clades each were determined for GOLS and RAFS.

FIG. 2 depicts southern blots of maize genomic DNA. The blots reveal greater homology among ZmGOLS genes than among ZmRAFS genes. Southern blots were probed with A) ZmGOLS or B) ZmRAFS cDNA probes. The blots were first washed at low stringency (2×SSC and 0.1% SDS, 65° C.; data not shown) and then at high stringency (0.1×SSC and 0.1% SDS, 65° C.). Asterisks are over bands unique to ZmGOLS1 while arrows indicate bands specific for and common to ZmGOLS2 and 3.

FIG. 3 depicts Northern blots used to characterize expression during different developmental periods. Genes encoding enzymes involved in RFO synthesis are induced by drying in maize seeds. Northern blots were used to characterize ZmGOLS and ZmRAFS1 gene expression during seed development, maize seed germination, and stress during imbibition. ZmRAFS2 and −3 mRNAs were not detected in maize seeds. Dehydration of 24 h imbibed seeds upregulated transcription of ZmGOLS and ZmRAFS1 genes.

FIG. 4 depicts the results of a ribonuclease protection assay. RPA analysis of maize seeds indicates temporal differences in the regulation of ZmGOLS gene expression. Ribonuclease protection assays of in vitro synthesized sense ZmGOLS RNAs, and RNA from dehydrated callus and imbibed-dehydrated seeds probed with: A) ZmGOLS1 antisense probe; B) ZmGOLS2 antisense probe; and C) ZmGOLS3 antisense probe. With one exception, only transcript from ZmGOLS2 was detectable in callus and seed RNA. However, RNA collected from unimbibed, mature, dehydrated seed D) probed with ZmGOLS2 antisense probe, contained only ZmGOLS3 transcript.

FIG. 5 depicts galactinol synthase enzyme activity and sugar content from seeds. Galactinol synthase enzyme activity and sugar contents from maize seeds do not exhibit a consistent, positive correlation. GOLS enzyme activity A) and seed raffinose content B) during maize seed germination, and stress during imbibition, were not consistently related. HAI: hours after imbibition at 25° C.

FIG. 6 depicts the local expression of ZmGOLS2 and ZmRAFS1. Tissue prints localize both ZmGOLS2 and ZmRAFS1 to the embryo of maize seeds. Maize seed tissue prints were made on nylon membranes, each half of a bisected seed being printed on a separate membrane and probed with equal specific activities (estimated from dot blots) of either the sense or antisense, DIG-labeled probes for ZmRAFS1 or ZmGOLS2. The bar in the first panel of ZmRAFS1 represents 1 mm.

FIG. 7 depicts Northern blots and RPA of total ZmGOLS RNA from Maize Hi-II cells. A) Maize cells were cultured on N6 media amended with 1) sucrose (Suc) (5%), 2) sucrose and raffinose (Raf) (2% each), 3) glucose (Glc) and raffinose (2% each), 4) 2% sucrose and myo-Inositol (myo-I) (0.01%), or 5) 2% sucrose and 7.8% mannitol (Man). After 8 d 20 h, maize cells were dehydrated in a flow hood for 4 h (Dehydrated) or, after 8d, maize cells were moved to 4 or 42° C. for 1 d. Each northern blot (20 μg per lane) was hybridized with α-³²P labeled cDNA probes to each ZmGOLS cDNA. B) Cells were dehydrated after culture on N6 media containing 2% (w/v) sucrose with no, low (0.1% w/v), medium (0.5% w/v), or high (1% w/v) myo-inositol or mannitol. Low myo-inositol stimulated while high myo-inositol inhibited ZmGOLS2 transcription relative to the 2% sucrose control. Low mannitol repressed ZmGOLS transcription but high mannitol had no effect on transcription relative to the 2% sucrose control. C) Mannitol and myo-inositol were added singly or in combination to media to determine if they acted synergistically to control ZmGOLS transcription during dehydration stress in maize callus cells. High mannitol and low myo-inositol or high mannitol and low scyllo-inositol (scy-I), provided together, were less stimulatory than high mannitol alone. Scyllo-inositol is a naturally occurring isomer of myo-inositol used here as a negative control.

FIG. 8 shows that ZmRAFS1 is upregulated by sucrose and heat stress in maize Hi-II callus.

A) Maize cells were cultured on N6 media amended with 1) sucrose (Suc) (5%), 2) sucrose and raffinose (Raf) (2% each), 3) glucose (Glc) and raffinose (2% each), 4) 2% sucrose and myo-inositol (myo-I) (0.01%), or 5) 2% sucrose and 7.8% mannitol (Man). After 8 d and 20 h, maize cells were dehydrated in a flow hood for 4 h (Dehydrated) or, after 8 d, maize cells were moved to 4 or 42° C. for 1 d. ZmRAFS1 expression was most sensitive to heat stress and was upregulated by sucrose. Each northern blot (20 μg per lane) was hybridized with α-³²P labeled cDNA probes to each ZmRAFS cDNA. Only ZmRAFS1 produced a detectable signal.

B) Cells were grown on N6 media supplemented with 2% (w/v) glucose (Glc2), 2% fructose (Frc2), glucose and fructose (1% each), or 2% or 4% sucrose (Suc2 and Suc4) and then stressed at high temperature. ZmRAFS1 was upregulated by sucrose in heat-stressed callus. As the osmotic potential of 2% monosaccharides was equal to that of 4% sucrose, osmotic stress was not responsible for greater ZmRAFS1 transcription on 4% sucrose.

C) Maize cells were cultured on N6 media amended with 2% fructose (Frc2), 2% glucose (Glc2), 2% glucose+0.05% sucrose (Suc0.05), 1.5% glucose+0.5% sucrose, 2% sucrose, 4% sucrose, 4% sucrose+0.01% raffinose (Raf0.01), 4% sucrose+0.1% raffinose, 4% sucrose+1% raffinose, 4% sucrose+0.33% galactose (Gal0.33), 4% sucrose+0.66% melibiose (Mel0.66), or 4% sucrose+0.33% fructose. After 8 d, maize cells were moved to 42° C. for 1 d. The membrane was hybridized with α³²P-labeled cDNA probe to ZmRAFS1. ZmRAFS2 and 3 gene expression was not detected in maize callus.

FIG. 9 demonstrates that Transcription of neither ZmRAFS1 nor ZmGOLS2 is induced by ABA in Maize Hi-II cells. Maize cells were cultured for 1 d on N6 media, 2% sucrose, with (ZmGOLS) or without (ZmRAFS1) 1% w/v mannitol, or N6 media, 2% sucrose supplemented with 1 μM, 10 μM, or 100 μM ABA. Mannitol-fed calli were dehydrated in a flow hood for 4 h (ZmGOLS positive control) while callus on 2% sucrose with (experimental) or without (ZmRAFS1 positive control) added ABA were not. Northern blots of total RNA (20 μg per lane) were hybridized with α-³²p labeled cDNA probes to each ZmGOLS and ZmRAFS cDNA. None of the ZmGOLS cDNAs hybridized to a detectable signal on ABA (ZmGOLS2 depicted) while only ZmRAFS1 produced detectable signal in callus cells.

FIG. 10 is a depiction of a model of the regulation of raffinose accumulation in maize seeds. Using data on ZmGOLS and ZmRAFS expression, sugar amounts, and their interaction under a variety of environmental stresses either collected in this project or obtained from the literature, a tentative model of the regulation of genes encoding raffinose biosynthetic enzymes has been developed. This model assumes that ZmGOLS3 expression in maturing seeds exhibits the same response to myo-inositol and dehydration that was documented for ZmGOLS2 in maize callus cells.

FIG. 11 demonstrates the results of a cold stress test performed on a RAFS1 line.

FIGS. 12a, 12b are bar graphs depicting the difference in height and number of green leaves between treatment groups observed in addition to stress response.

FIG. 13 is a bar graph depicting maize plant height of Treatment C.

FIG. 14 shows a plot of weight loss over time.

FIG. 15 shows a plot of conductance rates over time.

### DETAILED DESCRIPTION OF THE INVENTION

The present invention provides, inter alia, compositions and methods for promoting abiotic and other stress tolerance and/or manipulating raffinose production in plants. As used herein, the term “abiotic stress” or “Abiotic stress condition” refers to the exposure of a plant, plant cell, or the like, to a non-living (“abiotic”) physical or chemical agent or condition that has an adverse effect on metabolism, growth, development, propagation and/or survival of the plant (collectively “growth”). An abiotic stress can be imposed on a plant due, for example, to an environmental factor such as water (e.g., flooding, drought, dehydration), anaerobic conditions (e.g., a low level of oxygen), abnormal osmotic conditions, salinity or temperature (e.g., hot/heat, cold, freezing, frost), a deficiency of nutrients or exposure to pollutants, or by a hormone, second messenger or other molecule. Anaerobic stress, for example, is due to a reduction in oxygen levels (hypoxia or anoxia) sufficient to produce a stress response. A flooding stress can be due to prolonged or transient immersion of a plant, plant part, tissue or isolated cell in a liquid medium such as occurs during monsoon, wet season, flash flooding or excessive irrigation of plants, or the like. A cold stress or heat stress can occur due to a decrease or increase, respectively, in the temperature from the optimum range of growth temperatures for a particular plant species. Such optimum growth temperature ranges are readily determined or known to those skilled in the art. Dehydration stress can be induced by the loss of water, reduced turgor, or reduced water content of a cell, tissue, organ or whole plant. Drought stress can be induced by or associated with the deprivation of water or reduced supply of water to a cell, tissue, organ or organism. Saline stress (salt stress) can be associated with or induced by a perturbation in the osmotic potential of the intracellular or extracellular environment of a cell. Osmotic stress also can be associated with or induced by a change, for example, in the concentration of molecules in the intracellular or extracellular environment of a plant cell, particularly where the molecules cannot be partitioned across the plant cell membrane.

The methods will provide for improved plant response to stressors such as heat, cold, salt or desiccation of susceptible plants. The compositions of the invention include nucleic acid molecules comprising sequences which are up or down regulated in response to abiotic stress. These compositions can be transferred into plants to confer or improve stress tolerance in transformed plants. By “confer or improve stress tolerance” is intended that the proteins or sequences, either alone or in combination with other proteins or sequences, enhance the ability of a plant to respond to an environmental and other abiotic stressors such as salinity, drought, cold, heat, mineral deficiency or mineral toxicity, or to help a plant respond to internal stress such as developmental periods that are particularly taxing on the resources of a plant such as seed development or to manipulate raffinose production of a plant or to respond to other stressors which cause a similar plant reaction. In this manner, resistance to environmental or other stress can be enhanced or improved in the transformed plant when at least one of the sequences of the invention is provided.

The availability of reverse genetics systems, which are well-known in the art, makes the generation and isolation of down-regulated or null mutants feasible, given the availability of a defined nucleic acid sequence, as provided herein. One such system (the Trait Utility System for Corn, i.e., TUSC) is based on successful systems from other organisms (Ballinger et al., Proc. Natl. Acad. Sci. USA, 86, 9402-9406 (1989); Kaiser et al. (1990), Proc. Natl. Acad. Sci. USA, 87, 1686-1690; and Rushforth et al., Mol. Cell. Biol., 13, 902-910 (1993)). The central feature of the system is to identify Mu transposon insertions within a DNA sequence of interest in anticipation that at least some of these insertion alleles will be mutants. See U.S. Pat. Nos. 6,300,542 and 5,962,764. To develop the system, DNA was collected from a large population of Mutator transposon stocks that were then self-pollinated to produced F2 seed. To find Mu transposon insertions within a specific DNA sequence, the collection of DNA samples is screened via PCR using a gene-specific primer and a primer that anneals to the inverted repeats of Mu transposons. A PCR product is expected only when the template DNA comes from a plant that contains a Mu transposon insertion within the target gene. Once such a DNA sample is identified, F2 seed from the corresponding plant is screened for a transposon insertion allele. Transposon insertion mutations of the an1 gene have been obtained via the TUSC procedure (Bensen et al. (1995)). This system is applicable to other plant species, at times modified in accordance with knowledge and skills reasonably attributed to ordinary artisans.

The compositions comprise nucleic acid molecules comprising sequences of plant genes and the polypeptides encoded thereby. Particularly, the nucleotide and amino acid sequences for a maize galactinol synthase (GOLS) and/or raffinose synthase (RAFS) are provided. Three GOLS encoding nucleotide encoding sequences are provided at SEQ ID NOS:1, 3, and 5 with the corresponding proteins at SEQ ID NOS: 2, 4, and 6. Three RAFS encoding nucleotide sequences are provided at SEQ ID NOS: 7, 9, and 11 with the corresponding proteins at SEQ ID NOS: 8, 10, and 12. As discussed in more detail below, the sequences of the invention are involved in many basic biochemical pathways that regulate plant stress tolerance. Thus, methods are provided for the expression of these sequences in a host plant to modulate plant stress responses. Some of the methods involve stably transforming a plant with a nucleotide sequence capable of modulating the plant metabolism operably linked with a promoter capable of driving expression of a gene in a plant cell.

The compositions also comprise nucleic acid molecules comprising sequences useful in the control of gene expression in improving stress tolerance. Promoter and other regulatory elements which are natively associated with these genes can be easily isolated using the sequences and methods described herein with no more than routine experimentation. These sequences can be used to identify promoter, enhancer or other signaling signals in the regulatory regions of these gene sequences. These regulatory and promoter elements provide for temporal and spatial expression of operably linked sequences with stress and/or raffinose production, and/or seed development in a plant. Methods are provided for the regulated expression of a nucleotide sequence of interest that is operably linked to the promoter regulatory sequences disclosed herein. Nucleotide sequences operably linked to the promoter sequences are transformed into a plant cell. Exposure of the transformed plant to a stimulus such as stress or the timing of seed development induces transcriptional activation of the nucleotide sequences operably linked to these promoter regulatory sequences.

The promoter sequences of the invention may find use in the regulated expression of an operably linked heterologous gene of interest. For example, the provided sequences may find use as a stress responsive promoter. In addition to these promoter sequences, the stress-regulated promoters of the invention encompass fragments and variants of these particular promoters as defined herein. Thus, a fragment of the promoter may be used either alone or in combination with other sequences to create synthetic promoter constructs. In such embodiments, the fragments (also called “cis-acting elements” or “subsequences”) confer desired properties on the synthetic promoter construct, such as conferring increased transcription of operably linked sequences in response to stress or seed development.

By “stress-regulated” promoter is intended a promoter whose transcription initiation activity is either induced or repressed in response to a stress stimulus or to seed development. Thus, a stress-inducible promoter increases expression of an operably linked nucleotide sequence in the presence of plant stress including but not limited to drought, salinity, cold, heat, mineral deficiency, or mineral toxicity. In contrast, a stress repressible promoter decreases the transcription of an operably linked nucleotide sequence in the presence of plant stress or seed development. Stress-repressible promoters provide a means for improved regulation of genetically engineered stress tolerance in plants. In addition to these promoter sequences, the stress-regulated promoters of the invention encompass fragments and variants of these particular promoters as defined herein.

Plant “stress” includes any biotic or abiotic influence on a plant which impedes plant growth. This includes but is not limited to: drought, increased salinity of the soil, cold temperatures, hot temperatures, mineral deficiency, and/or mineral toxicity. Other examples of sources of plant stress contemplated in the present invention are given elsewhere herein.

By “heterologous nucleotide sequence” is intended a sequence that is not naturally occurring with the promoter sequence. While this nucleotide sequence is heterologous to the promoter sequence, it may be homologous, or native, or heterologous, or foreign, to the plant host. By “operably linked” is intended a functional linkage between a promoter sequence and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

The type of nucleotide sequence within a nucleic acid molecule of the invention depends upon its intended use. Thus, when the nucleic acid molecule comprises a stress-inducible promoter, it is of interest to operably link that promoter to a nucleotide sequence useful in improving stress tolerance. Such sequences are referred to herein as “stress tolerance sequence.” By “stress tolerance sequence” is intended a sequence coding for an RNA and/or a protein or polypeptide that, when expressed, either inhibits, prevents, or otherwise favorably alters a plants response to stress to improve growth, yield, or overall health of a plant. Possible sources of stress include but are not limited to drought, salinity, cold, heat, mineral deficiency, mineral toxicity, or periods of plant development such as seed development which are demanding on a plant. Such sequences include sequences encoding stress responsive proteins or regulatory proteins that associated with the stress response in plants. Expression of such sequences allows a plant to avoid the symptoms associated with stress, or prevent or minimize the stress response from causing associated symptoms.

Structural genes employed in carrying out the present invention include any sequence the expression of which is desired in a plant cell during periods of stress. This includes enzymes involved in carbohydrate synthesis pathways of a plant or any other protein which is beneficial to a plant in conditions of stress.

Nucleic acid sequences encoding gene products useful in improving a plants response to stress are provided. Particularly, a nucleic acid sequence encoding a proteins involved in the production of carbohydrates in plants, namely galactinol synthase and raffinose synthase are provided.

The galactinol synthase, raffinose synthase, proteins and their promoter/regulatory regions form a part of the plant's response to attack stress. Thus the sequences of the invention find use in controlling or modulating gene expression as well as the response to stress.

Transformed plants can be obtained which have altered or enhanced responses to stress, thus, the sequences of the invention find use in engineering broad-spectrum stress tolerance in a variety of plants. A polypeptide is said to have GOLS or RAFS-like activity when it has one or more of the properties of the native protein. It is within the skill in the art to assay protein activities obtained from various sources to determine whether the properties of the proteins are the same. In so doing, one of skill in the art may employ any of a wide array of known assays including, for example, biochemical and/or pathological assays. For example, one of skill in the art could readily produce a plant transformed with a GOLS polypeptide variant and assay a property of native GOLS protein in that plant material to determine whether a particular GOLS property was retained by the variant.

The compositions and methods of the invention are involved in biochemical pathways and as such may also find use in the activation or modulation of expression of other genes, including those involved in other aspects of stress response. For example, in one embodiment of the invention, the maize GOLS promoter is used to drive expression of restorative proteins which are accordingly induced in response to plant dehydration.

Although there is some conservation among these genes, proteins encoded by members of these gene families may contain different elements or motifs or sequence patterns that modulate or affect the activity, subcellular localization, and/or target of the protein in which they are found. Such elements, motifs, or sequence patterns may be useful in engineering novel enzymes for modulating gene expression in particular tissues. By “modulating” or “modulation” is intended that the level of expression of a gene may be increased or decreased relative to genes driven by other promoters or relative to the normal or uninduced level of the gene in question.

According to the invention, RAFS encoding nucleotide sequences were induced by all stresses, while GOLS encoding sequences were induced only by desiccation. Expression of the proteins encoded by the sequences of the invention can be used to modulate or regulate the expression of proteins in these stress-response pathways and other directly or indirectly affected pathways. Hence, the compositions and methods of the invention find use in altering plant response to the environment and environmental stimuli. In other embodiments, fragments of the genes are used to confer desired properties to synthetic protein constructs for use in regulating plant growth or cellular processes, such as seed development.

The present invention provides for isolated nucleic acid molecules comprising nucleotide sequences encoding the amino acid sequence shown in SEQ ID NOS: 2, 4, 6, 8, and 12, as well as their conservatively modified variants or the nucleotide sequences of the nucleic acid molecules deposited in a bacterial host as Patent Deposit No. ______. Further provided are polypeptides having an amino acid sequence encoded by a nucleic acid molecule described herein, for example those polypeptides comprising the sequences set forth in SEQ ID NO: 1, 3, 5, 7, 9, and 11 or those deposited in a bacterial host as Patent Deposit Nos. ______, and fragments and variants thereof.

The present invention further provides for an isolated nucleic acid molecule comprising the sequences shown in SEQ ID NO: 1, 3, 5, 7, 9, or 11 or the nucleotide sequences deposited in a bacterial host as Patent Deposit No. ______.

Plasmids containing the nucleotide sequences of the invention were deposited with the Patent Depository of the American Type Culture Collection (ATCC), Manassas, Va., and assigned Patent Deposit No. ______. These deposits will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. § 112.

The invention encompasses isolated or substantially purified nucleic acid or protein compositions. An “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In some embodiments, an “isolated” nucleic acid is free of sequences (such as other protein-encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, 0.4 kb, 0.3 kb, 0.2 kb, or 0.1 kb, or 50, 40, 30, 20, or 10 nucleotides that naturally flank the nucleic acid molecule in genomic DNA of the cell from which the nucleic acid is derived. A protein that is substantially free of cellular material includes preparations of protein having less than about 30%, 20%, 10%, 5%, (by dry weight) of contaminating protein. When the protein of the invention or biologically active portion thereof is recombinantly produced, culture medium may represent less than about 30%, 20%, 10%, or 5% (by dry weight) of chemical precursors or non-protein-of-interest chemicals.

Fragments and variants of the disclosed nucleotide sequences are encompassed by the present invention. Fragments and variants of proteins encoded by the disclosed nucleotide sequences are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the native protein and hence affect development, developmental pathways, stress responses, and/or seed development by retaining GOLS or RAFS-like activity. Alternatively, fragments of a nucleotide sequence that are useful as hybridization probes generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the proteins of the invention.

A fragment of a GOLS or RAFS nucleotide sequence that encodes a biologically active portion of a GOLS or RAFS protein of the invention will encode at least 12, 25, 30, 50, 75, 100, 125, 150, 175, 200, 225, 250, 275, 300, 325, 350, 375, 400, 425, 450, 475, 500, 525, 550, 575, 600, 625, 650, 675, or 680 contiguous amino acids, or up to the total number of amino acids present in a full-length GOLS or RAFS protein of the invention.

Fragments of a GOLS or RAFS nucleotide sequence that are useful as hybridization probes or PCR primers generally need not encode a biologically active portion of a protein. Thus, a fragment of a GOLS or RAFS nucleotide sequence may encode a biologically active portion of a GOLS or RAFS protein, or it may be a fragment that can be used as a hybridization probe or PCR primer using methods disclosed below. A biologically active portion of a GOLS or RAFS protein can be prepared by isolating a portion of the GOLS or RAFS nucleotide sequences of the invention, expressing the encoded portion of the GOLS or RAFS protein (e.g., by recombinant expression in vitro), and assessing the activity of the encoded portion of the GOLS or RAFS protein. Nucleic acid molecules that are fragments of a GOLS or RAFS nucleotide sequence comprise at least 16, 20, 25, 30, 40, 50, 60, 70, 80, 90, 100, 125, 150, 175, 200, 225, 250, 300, 350, 400, 450, 500, 550, 600, 650, 700, 750, 800, 850, 900, 950, 1000, 1050, 1100, 1200, 1300, 1400, 1500, 1600, 1700, 1800, 1900, 2000, 2100, 2200, 2300, or 2400 nucleotides, or up to the number of nucleotides present in a full-GOLS or RAFS nucleotide sequences disclosed herein.

By “variants” is intended substantially similar sequences. For nucleotide sequences, conservative variants include those sequences that, because of the degeneracy of the genetic code, encode the amino acid sequence of one of the polypeptides of the invention. Naturally occurring allelic variants such as these can be identified with the use of well-known molecular biology techniques, as, for example, with polymerase chain reaction (PCR) and hybridization techniques as outlined below. Variant nucleotide sequences also include synthetically-derived nucleotide sequences, such as those generated, for example, by using site-directed mutagenesis but which still encode a GOLS or RAFS protein of the invention. Generally, variants of a particular nucleotide sequence of the invention will have at least 40%, 50%, 60%, 70%, generally at least 75%, 80%, 85%, or about 90%, 91%, 92%, 93%, 94%, 95%, 96%,97%, 98%, 99%, or more sequence identity to that particular nucleotide sequence as determined by sequence alignment programs described elsewhere herein using default parameters.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is they continue to possess the desired biological activity of the native protein, hence they will continue to possess at least one activity possessed by the native GOLS or RAFS protein. Such variants may result from, for example, genetic polymorphism or from human manipulation. Biologically active variants of a GOLS or RAFS native protein of the invention will have at least 40%, 50%, 60%, 70%, generally at least 75%, 80%, 85%, 86%, 87%, 88%, 89%, about 90%, 91%, 92%, 93%, 94%, 95%, 96%, 97%, 98%, 99%, or more sequence identity to the amino acid sequence for the native protein as determined by sequence alignment programs described elsewhere herein using default parameters. A biologically active variant of a protein of the invention may differ from that protein by as few as 1-15 amino acid residues, as few as 1-10, , as few as 5, as few as 4, 3, 2, or even 1 amino acid residue. As used herein, reference to a particular nucleotide or amino acid sequence (a GOLS or FAFS sequence) shall include all modified variants as described supra.

The proteins of the invention may be altered in various ways including amino acid substitutions, deletions, truncations, and insertions. Methods for such manipulations are generally known in the art. For example, amino acid sequence variants of GOLS or RAFS proteins can be prepared by mutations in the DNA. Methods for mutagenesis and nucleotide sequence alterations are well known in the art. See, for example, Kunkel (1985) Proc. Nad. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be made.

Thus, the genes and nucleotide sequences of the invention include both naturally occurring sequences as well as mutant forms. Likewise, the proteins of the invention encompass both naturally-occurring proteins as well as variations and modified forms thereof. Such variants will continue to possess the desired GOLS or RAFS-like activity. It is recognized that variants need not retain all of the activities and/or properties of the native GOLS or RAFS protein. Obviously, the mutations that will be made in the DNA encoding the variant must not place the sequence out of reading frame and in some embodiments will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity of GOLS or RAFS polypeptides can be evaluated by either an enhanced response to stress or a modulation in a plant developmental or metabolic process when expression of the protein or polypeptide sequence is altered. For example, GOLS or RAFS-like activity may be evaluated as a change in gene transcription in genes downstream from GOLS or RAFS in the stress-response pathway in the plant.

Variant nucleotide sequences and proteins also encompass sequences and proteins derived from a mutagenic and recombinogenic procedure such as DNA shuffling. With such a procedure, one or more different GOLS or RAFS coding sequences can be manipulated to create a new GOLS or RAFS possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the GOLS or RAFS gene of the invention and other known GOLS or RAFS genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389391; Crameri et al. (1997) Nature Biotech. 15:436-438; Moore et al. (1997) J. Mol. Biol. 272:336-347; Zhang et al. (1997) Proc. Natl. Acad. Sci USA 94:4504-4509; Crameri et al. (1998) Nature 391:288-291; and U.S. Pat. Nos. 5,605,793 and 5,837,458.

The compositions of the invention also include isolated nucleic acid molecules comprising the promoter nucleotide sequences natively associated with these polynucleotides. By “promoter” is intended a regulatory region of DNA usually comprising a TATA box capable of directing RNA polymerase II to initiate RNA synthesis at the appropriate transcription initiation site for a particular coding sequence. A promoter may additionally comprise other recognition sequences generally positioned upstream or 5′ to the TATA box, referred to as upstream promoter elements, which influence the transcription initiation rate.

It is recognized that having identified the nucleotide sequences for the promoter regions disclosed herein, it is within the state of the art to isolate and identify additional regulatory elements in the 5′ untranslated region upstream from the particular promoter regions defined herein. Thus for example, the promoter regions disclosed herein may further comprise upstream regulatory elements that confer tissue-preferred expression of heterologous nucleotide sequences operably linked to the disclosed promoter sequence. See particularly, Australian Patent No. AU-A-77751/94 and U.S. Pat. Nos. 5,466,785 and 5,635,618. It is also recognized by those of skill in the art that regulatory elements may be found in transcribed regions of a gene, for example in the region between transcription start and translation start as well as 3′ to the end of translation; such elements may be found in the sequences set forth herein. Regulatory elements, as used herein, may also be found within the coding region itself.

Fragments and variants of the disclosed promoter sequences are also encompassed by the present invention. By “fragment” is intended a portion of the nucleotide sequence. Fragments of a nucleotide sequence may retain biological activity and hence retain their transcriptional regulatory activity. Thus, for example, less than the entire promoter sequence disclosed herein may be utilized to drive expression of an operably linked nucleotide sequence of interest, such as a nucleotide sequence encoding a heterologous stress-resistance polypeptide. Alternatively, fragments of a nucleotide sequence that are useful as hybridization probes generally do not retain biological activity. Thus, a fragment of GOLS or RAFS promoter nucleotide sequence may encode a biologically active portion of the GOLS or RAFS promoter, or it may be a fragment that can be used as a hybridization probe or PCR primer using methods disclosed below. A biologically active portion of a GOLS or RAFS promoter can be prepared by isolating a portion of one of GOLS or RAFS promoter nucleotide sequences of the invention, and assessing the activity of the portion of the GOLS or RAFS promoter. Nucleic acid molecules that are fragments of a GOLS or RAFS promoter nucleotide sequence comprise at least about 16 to 20 nucleotides to about 30, 40, 50, 60, 70, 80, 90, 100, 150, 200, 250, 300, 350, 400, 450, 500, 550, 600, 650, 700, 750, 800, 850, 900, 950, 1000, 1050, 1100, 1200, 1300, 1400, 1500, 1600, 1700, 1800, 1900, or 2000 nucleotides, or up to the number of nucleotides present in a full-length GOLS or RAFS nucleotide sequence disclosed herein.

Fragment lengths depend upon the objective and will also vary depending upon the particular promoter sequence. Thus, where the promoter fragment is to be used as a functional promoter, suitable promoter fragments or variants retain functional promoter activity, that is, the fragments or variants obtained are capable of directing RNA polymerase II to initiate RNA synthesis at the appropriate transcription initiation site for a particular coding sequence in response to a stress stimulus where the promoter is stress-inducible or direct transcription in the absence of the stimulus in the case of a stress-repressible promoter. It is within the skill in the art to determine whether such fragments decrease expression levels or alter the nature of expression, i e., stress-inducible or stress-repressible expression, and assays to determine the activity of a promoter sequence are well known in the art. For example, the production of RNA transcripts may be assayed by Northern blot hybridization. Alternatively, a GOLS or RAFS promoter fragment or variant may be operably linked to the nucleotide sequence encoding any reporter protein, such as the (3-glucuronidase protein (GUS reporter) or the luciferase protein or the like. The DNA construct may inserted into the genome of a plant or plant cell and the mRNA or protein levels of the reporter sequence determined. See, for example, Eulgem et al. (1999) EMBO Journal 18: 4689-4699; U.S. Pat. No. 6,072,050, herein incorporated by reference.

By promoter “variants” is intended promoter sequences having substantial similarity with a synthetic promoter sequence disclosed herein. Generally, variants of a particular nucleotide sequence of the invention will have at least about 40%, 50%, 60%, 65%, 70%, generally at least about 75%, 80%, 85%, 86%, 87%, 88%, 89%, at least about 90%, 91%, 92%, 93%, 94%, 95%, 96%, 97%, or at least about 98%, 99% or more sequence identity to that particular nucleotide sequence as determined by sequence alignment programs described elsewhere herein using default parameters. Thus, variants may differ by only a few nucleotides, such as 50, 25, 20, 15, 10, 9, 8, 7, 6, 5, 4, 3, 2, or even 1 nucleotide. Such variants retain the stress-regulated promoter activity of the disclosed promoter sequences. Thus variants of the GOLS or RAFS sequence retain stress-inducible promoter activity.

The variant promoter sequences will share substantial homology with their corresponding synthetic promoter sequence. By “substantial homology” is intended a sequence exhibiting substantial functional and structural equivalence with the disclosed sequence. Any functional or structural differences between substantially homologous sequences do not effect the ability of the sequence to function as a stress-regulated promoter. Thus, for example, any sequence having substantial sequence homology with the sequence of a particular stress-inducible promoter of the present invention will direct expression of an operably linked heterologous nucleotide sequence in response to a stress stimulus. Two nucleotide sequences are considered substantially homologous when they have at least about 50%, 60%, 65%, 70%, 73%, 75%, 78%, 80%, 81%, 82%, 83%, 84%, 85%, 86%, 87%, 88%, 89%, 90%, 91%, 92%, 93%, 94%, 95%, 96%, 97%, to 98% or 99% or higher sequence homology. Substantially homologous sequences of the present invention include variants of the disclosed sequences such as those that result from site-directed mutagenesis, as well as synthetically derived sequences.

The nucleotide sequences of the invention can be used to isolate corresponding sequences from other organisms, particularly other plants, more particularly other crop plants. In this manner, methods such as PCR, hybridization, and the like can be used to identify such sequences based on their sequence homology to the sequences set forth herein. Sequences isolated based on their sequence identity to the nucleotide sequences set forth herein or to fragments thereof are encompassed by the present invention. Such sequences include sequences that are orthologs of the disclosed sequences. By “orthologs” is intended genes derived from a common ancestral gene and which are found in different species as a result of speciation. Genes found in different species are considered orthologs when their nucleotide sequences and/or their encoded protein sequences share substantial identity as defined elsewhere herein. Functions of orthologs are often highly conserved among species. Thus, isolated sequences that have GOLS or RAFS promoter activity or encode a GOLS or RAFS protein and which hybridize under stringent conditions to GOLS or RAFS sequences disclosed herein, or to fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present it a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the disease-resistant sequences of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire sequence disclosed herein, or one or more portions thereof, may be used as a probe capable of specifically hybridizing to corresponding stress-response sequences, including promoters and messenger RNAs. To achieve specific hybridization under a variety of conditions, such probes include sequences that are unique among stress-response sequences and may be at least about 10 or 15 or 17 nucleotides in length or at least about 20 or 22 or 25 nucleotides in length. Such probes may be used to amplify corresponding sequences from a chosen organism by PCR. This technique may be used to isolate additional coding sequences from a desired organism or as a diagnostic assay to determine the presence of coding sequences in an organism. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different under different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length or less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3. Incubation should be at a temperature of least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl, 0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours.

Specificity is typically a function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), the SSC concentration may be increased so that a higher temperature can be used. An extensive guide to the hybridization of nucleic acids is found in Tijssen (1993) Laboratory Techniques in Biochemistry and Molecular Biology-Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

In general, sequences that have promoter activity or encode a GOLS or RAFS protein and which hybridize to the GOLS or RAFS sequences disclosed herein will be at least about 40% homologous, about 50% or 60% homologous, about 70% homologous, and even about 80%, 85%, 90%, 91%, 92%, 93%, 94%, 95%, 98%, 99% or more homologous with the disclosed sequences. That is, the sequence identity of the sequences may be from about 40% to 50% identical, about 60% to 70% or 75%, and even about 80%, 85%, 87%, 90%, 91%, 92%, 93%, 94%, 95%, 96%, 97%, 98%, or 99% identical, or higher, so that the sequences may differ by only 10, 9, 8, 7, 6, 5, 4, 3, 2, or 1 amino acid residue or by 20, 19, 18, 17, 16, 15, 14, 13, 12, 11, 10, 9, 8, 7, 6, 5, 4, 3, 2, or 1 nucleic acid.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, or PSI-BLAST, the default parameters of the respective programs (e.g., BLASTN for nucleotide sequences, BLASTX for proteins) can be used (see information at www.ncbi.nlm.nih.gov). Alignment may also be performed manually by inspection.

Unless otherwise stated, sequence identity/similarity values provided herein refer to the value obtained using GAP version 10 using the following parameters: % identity using GAP Weight of 50 and Length Weight of 3; % similarity using Gap Weight of 12 and Length Weight of 4, or any equivalent program. By “equivalent program” is intended any sequence comparison program that, for any two sequences in question, generates an alignment having identical nucleotide or amino acid residue matches and an identical percent sequence identity when compared to the corresponding alignment generated by GAP Version 10. GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff(1989) Proc. Natl. Acad. Sci. USA 89:10915).

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 80%, 85%, 90%, 95%, or higher sequence identity compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 50%, 55%, 60%, 65%, 70%, 75%, 80%, 85%, 90%, or at least 95% or higher sequence identity.

Another indication that nucleotide sequences are substantially identical is if two molecules hybridize to each other under stringent conditions. Generally, stringent conditions are selected to be about 5° C. lower than the thermal melting point (Trr,) for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross-reactive with the polypeptide encoded by the second nucleic acid.

(e)(ii) The term “substantial identity” in the context of a peptide indicates that a peptide comprises a sequence with at least 70%, 75%, 80%, 83%, 85%, 88%, 90%, 93%, 95%, 96%, 97%, 98%, or 99% or higher sequence identity to the reference sequence over a specified comparison window. Preferably, optimal alignment is conducted using the homology alignment algorithm of Needleman and Wunsch (1970) J Mol. Biol. 48:443453. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

Methods for increasing stress tolerance in a plant are provided. In some embodiments, the methods involve stably transforming a plant with a DNA construct comprising an stress responsive nucleotide sequence of the invention operably linked to a promoter that drives expression in a plant. While the choice of promoter will depend on the desired timing and location of expression of the stress-responsive or other nucleotide sequences, desirable promoters include constitutive and pathogen-inducible promoters. In some embodiments, such a promoter will be a GOLS or RAFS promoter of the invention, as further discussed below. These methods may find use in agriculture, particularly in limiting the impact of abiotic stress on crop plants. Thus, transformed plants, plant cells, plant tissues and seeds thereof are provided by the present invention.

Additionally, the compositions of the invention can be used in formulations for their disease resistance activities. The proteins of the invention can be formulated with an acceptable carrier into a pesticidal or nematicidal composition(s) that is, for example: a suspension, a solution, an emulsion, a dusting powder, a dispersible granule, a wettable powder, an emulsifiable concentrate, an aerosol, an impregnated granule, an adjuvant, a coatable paste, or an encapsulation in, for example, polymer substances.

It is understood in the art that plant DNA viruses and fungal pathogens remodel the control of the host replication and gene expression machinery to accomplish their own replication and effective infection. The plant response to stress, such as stress caused by drought, is known to involve many basic biochemical pathways and cellular functions. Hence, the sequences of the invention may find use in altering the mechanisms of a host plant to provide broad-based resistance to stress.

The present invention may be used in conjunction with one or more other methods to increase stress tolerance. In some embodiments of the invention, a second nucleotide sequence is transformed into a plant to increase the plant's resistance stress. In these embodiments, any one of a variety of second nucleotide sequences may be utilized. It is recognized that such second nucleotide sequences may be used in either the sense or antisense orientation.

In another embodiment, the methods of the present invention involve identifying phenotypes associated with a loss of raffinose synthase 1 (RAFS 1) activity in plants that contain transposon insertions within the RAFS 1 gene.

In other embodiments, the methods of the present invention involve stably transforming a plant with a DNA construct comprising a promoter of the invention linked to a nucleotide sequence which confers increased resistance to stress. In this manner, the GOLS or RAFS promoters disclosed herein may provide regulation of expression of operably linked coding regions to control stress. Additionally, GOLS or RAFS promoters disclosed herein are useful for genetic engineering of plants to express a phenotype of interest. The promoter sequences may be used to drive expression of any heterologous nucleotide sequence. Alternatively, GOLS or RAFS promoter sequence may be used to drive expression of its native, i.e., naturally occurring GOLS or RAFS gene sequence disclosed herein. In such an embodiment, the phenotype of the plant is altered. In some embodiments, GOLS or RAFS promoter sequences are operably linked to a nucleotide sequence and drive expression of said sequence in a plant cell. The GOLS or RAFS promoter sequences may therefore be used in creating or enhancing stress resistance in a transformed plant.

In some embodiments, the nucleic acid molecules comprising GOLS or RAFS sequences of the invention are provided in expression cassettes or DNA constructs for expression in the plant of interest. Such cassettes will include 5′ and 3′ regulatory sequences operably linked to a GOLS or RAFS sequence of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the GOLS or RAFS sequence to be under the transcriptional regulation of the regulatory regions. The expression cassette may additionally contain selectable marker genes. The expression cassette will include in the 5′-3′ direction of transcription, a transcriptional and translational initiation region, a GOLS or RAFS DNA sequence of the invention, and a transcriptional and translational termination region functional in plants. The transcriptional initiation region, or promoter, may be native or analogous or foreign or heterologous to the plant host. Additionally, the promoter may be the natural sequence or alternatively a synthetic sequence. By “foreign” is intended that the transcriptional initiation region is not found in the native plant into which the transcriptional initiation region is introduced. As used herein, a “chimeric gene” comprises a coding sequence operably linked to a transcription initiation region that is heterologous to the coding sequence.

While it may be preferable to express GOLS or RAFS sequences using heterologous promoters, the native promoter sequences may be used. Such constructs would change expression levels of the GOLS or RAFS protein in the plant or plant cell. Thus, the phenotype of the plant or plant cell is altered.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639. Where appropriate, the gene(s) may be optimized for increased expression in the transformed plant. That is, the genes can be synthesized using plant-preferred codons for improved expression. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well characterized sequences that may be deleterious to gene expression. The G-C content of the sequence may be adjusted to enhance expression in a given host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Nat'l. Acad. Sci. USA 86:6126-6130); potyvirus leaders, for example, TEV leader (Tobacco Etch Virus) (Allison et al. (1986); MDMV leader (Maize Dwarf Mosaic Virus); Virology 154:9-20), and human immunoglobulin heavy-chain binding protein (BiP), (Macejak et al. (1991) Nature 353:90-94); untranslated leader from the coat protein mRNA of alfalfa mosaic virus (AMV RNA 4) (Jobling et al. (1987) Nature 325:622625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In those instances where it is desirable to have the expressed product of the heterologous nucleotide sequence of interest directed to a particular organelle, such as the chloroplast or mitochondrion, or secreted at the cell's surface or extracellularly, the expression cassette may further comprise a coding sequence for a transit peptide. Such transit peptides are well known in the art and include, but are not limited to, the transit peptide for the acyl carrier protein, the small subunit of RUBISCO, plant EPSP synthase, and the like.

In preparing the expression cassette, the various DNA fragments may be manipulated so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Nad. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Nad. Acad. Sci. USA 86:5400-5404; Fuerst et al. (1989) Proc. Nad. Acad. Sci. USA 86:2549-2553; Deuschle et al. (1990) Science 248:480-483; Gossen (1993) Ph.D. Thesis, University of Heidelberg; Reines et al. (1993) Proc. Nad. Acad. Sci USA 90:1917-1921; Labow et al. (1990) Mol. Cell. Biol. 10:3343-3356; Zambretti et al. (1992) Proc. Nad. Acad. Sci. USA 89:3952-3956; Baim et al. (1991) Proc. Nad. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Nad. Acad Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention. A number of promoters can be used in the practice of the invention. The promoters can be selected based on the desired outcome. That is, the nucleic acids can be combined with constitutive, tissue-preferred, or other promoters for expression in plants. Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Generally, it will be beneficial to express the gene from an inducible promoter, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J Plant Pathol. 89:245-254; Uknes et al. (1992) Plant Cell 4:645-656; and Van Loon (1985) Plant Mol. Virol. 4:111-116. See also the copending applications entitled “Inducible Maize Promoters,” U.S. patent application Ser. No. 09/257,583, filed Feb. 25, 1999; Publication No. WO 99/43819, published Sep. 9, 1999, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to: the maize In2-2 promoter, which is activated by benzenesulfonamide herbicide safeners; the maize GST promoter, which is activated by hydrophobic electrophilic compounds that are used as pre-emergent herbicides; and the tobacco PR-1 a promoter, which is activated by salicylic acid. Other chemical-regulated promoters of interest include steroid-responsive promoters. See, for example, the glucocorticoid-inducible promoter in Schena et al. (1991) Proc. Nad. Acad. Sci. USA 88:10421-10425 and McNellis et al. (1998) Plant J. 14(2):247-257) and tetracycline-inducible and tetracycline-repressible promoters (for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced gene expression within a particular plant tissue. Tissue-preferred promoters include Yamamoto et al. (1997) Plant J 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression. Leaf-specific promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J 3:509-18; Orozco et al. (1993) Plant Mol. Biol. 23(6):1129-1138; and Matsuoka et al. (1993) Proc. Nad. Acad. Sci. USA 90(20):9586-9590.

Where low level expression is desired, weak promoters will be used. Generally, by “weak promoter” is intended a promoter that drives expression of a coding sequence at a low level. By low level is intended at levels of about 1/1000 transcripts to about 1/100,000 transcripts to about 1/500,000 transcripts per cell. Alternatively, it is recognized that weak promoters also include promoters that are expressed in only a few cells and not in others to give a total low level of expression. Where a promoter is expressed at unacceptably high levels, portions of the promoter sequence can be deleted or modified to decrease expression levels. Such weak constitutive promoters include, for example, the core promoter of the Rsyn7 promoter (WO 99/43838 and U.S. Pat. No. 6,072,050), the core 35S CaMV promoter, and the like. Other constitutive promoters include, for example, U.S. Pat. Nos. 5,608,149; 5,608,144; 5,604,121; 5,569,597; 5,466,785; 5,399,680; 5,268,463; and 5,608,142. See also, U.S. Pat. No.6,177,611, herein incorporated by reference.

As used herein, “vector” refers to a molecule such as a plasmid, cosmid or bacterial phage for introducing a nucleotide construct and/or expression cassette into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

The methods of the invention involve introducing a nucleotide construct into a plant. By “introducing” is intended presenting to the plant the nucleotide construct in such a manner that the construct gains access to the interior of a cell of the plant. The methods of the invention do not depend on a particular method for introducing a nucleotide construct to a plant, only that the nucleotide construct gains access to the interior of at least one cell of the plant. Methods for introducing nucleotide constructs into plants are known in the art including, but not limited to, stable transformation methods, transient transformation methods, and virus-mediated methods.

By “stable transformation” is intended that the nucleotide construct introduced into a plant integrates into the genome of the plant and is capable of being inherited by progeny thereof. By “transient transformation” is intended that a nucleotide construct introduced into a plant does not integrate into the genome of the plant.

The nucleotide constructs of the invention may be introduced into plants by contacting plants with a virus or viral nucleic acids. Generally, such methods involve incorporating a nucleotide construct of the invention within a viral DNA or RNA molecule. It is recognized that the GOLS or RAFS protein of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing nucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931, herein incorporated by reference.

A variety of other transformation protocols are contemplated in the present invention. Transformation protocols as well as protocols for introducing nucleotide sequences into plants may vary depending on the type of plant or plant cell, i. e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Nad. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, eds. Gamborg and Phillips (Springer-Verlag, Berlin); McCabe et al. (1988) Biotechnology 6:923-926); and Lecl transformation (WO 00/28058, published May 18, 2000). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Nad Acad. Sci. USA 85:43054309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Nad. Acad Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

The present invention may be used for transformation of any plant species, including, but not limited to, monocots and dicots. Examples of plant species of interest include, but are not limited to, corn (Zea mays), Brassica spp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thujaplicata) and Alaska yellow cedar (Chamaecyparis nootkatensis). Plants of the present invention may be crop plants (for example, alfalfa, sunflower, Brassica, cotton, safflower, peanut, sorghum, wheat, millet, tobacco, etc.), corn or soybean plants.

Plants of particular interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

It is recognized that with these nucleotide sequences, antisense constructions complementary to at least a portion of the messenger RNA (mRNA) for GOLS or RAFS sequences can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, 85%, 90%, 95% or more sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

The nucleotide sequences of the present invention may also be used in the sense orientation to suppress the expression of endogenous genes in plants. Methods for suppressing gene expression in plants using nucleotide sequences in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, such as greater than about 65%, 75%, 85%, 95%, or higher sequence identity. See, U.S. Pat. Nos. 5,283,184 and 5,034,323; herein incorporated by reference. Posttranscriptional gene silencing may also result from the presence of RNA or double-stranded RNA which is thought to trigger cell-mediated degradation of homologous RNAs. See, for example, Matzke et al. (2001) Curr. Op. Genet. Dev. 11:221-227.

The nucleotide sequences of GOLS or RAFS promoters disclosed in the present invention, as well as variants and fragments thereof, are useful in the genetic manipulation of any plant when assembled with a construct such that the promoter sequence is operably linked to a nucleotide sequence encoding a heterologous protein of interest. In this manner, the nucleotide sequences of the GOLS or RAFS promoters of the invention can be provided in expression cassettes along with heterologous nucleotide sequences for expression in the plant of interest. Such an expression cassette is provided with a plurality of restriction sites for insertion of the nucleotide sequence to be under the transcriptional regulation of the stress-regulated promoter region. The expression cassette may additionally contain selectable marker genes.

The expression cassette will include in the 5′-to-3′ direction of transcription a transcriptional and translational initiation region comprising the stress-regulated GOLS or RAFS promoter (or variant or fragment thereof), a nucleotide sequence of interest which may be a heterologous nucleotide sequence or a GOLS or RAFS sequence, and a transcriptional and translational termination region functional in plants. The termination region may be native with the transcriptional initiation region comprising one of the promoter nucleotide sequences of the present invention, may be native with the DNA sequence of interest, or may be derived from another source. Convenient termination regions are available from the Ti-plasmid of A. tumefaciens, such as the octopine synthase and nopaline synthase termination regions (see references cited herein above). An expression cassette comprising a GOLS or RAFS promoter sequence may also contain features or modifications as described herein above for expression cassettes comprising nucleotide sequences of a GOLS or RAFS coding region of the invention.

The expression cassette comprising GOLS or RAFS promoter sequence (or variant or fragment thereof) operably linked to a heterologous nucleotide sequence of interest may also contain at least one additional nucleotide sequence for a gene to be cotransformed into the organism. Alternatively, the additional sequence(s) can be provided on another expression cassette.

The promoter for the GOLS or RAFS gene may regulate expression of operably linked nucleotide sequences in an inducible manner. That is, expression of the operably linked nucleotide sequences in a plant cell is induced in response to a stimulus. By “stimulus” is intended: a chemical, which may be applied externally or may accumulate in response to another external stimulus; other stresses such as environmental stresses, including but not limited to drought, temperature, and salinity; or other factor such as a pathogen, which may, for example, induce expression as a result of invading a plant cell.

Synthetic promoters are known in the art. Such promoters comprise upstream promoter elements (also referred to as “fragments” or “subsequences”) of one nucleotide sequence operably linked to at least one promoter element of another nucleotide sequence. In an embodiment of the invention, heterologous gene expression is controlled by a synthetic hybrid promoter comprising the GOLS or RAFS promoter sequences of the invention, or a variant or fragment thereof, operably linked to upstream promoter element(s) from a heterologous promoter. These elements can be as small as 4 or 6 base pairs, and can regulate stress-responsive activity of other promoters by cloning one or more copies of the element into the promoters. Alternatively, a GOLS or RAFS promoter sequence may comprise duplications of upstream elements found within the GOLS or RAFS promoter sequence. In order to increase transcription levels, enhancers may be utilized in combination with the promoter regions of the invention. Enhancers are nucleotide sequences that act to increase the expression of a promoter region. Enhancers are known in the art and include the SV40 enhancer region, the 35S enhancer element, and the like.

It is recognized that the promoter sequence of the invention may be used with its GOLS or RAFS coding sequences. A DNA construct comprising the GOLS or RAFS promoter operably linked with its native GOLS or RAFS gene may be used to transform any plant of interest to bring about a desired phenotypic change, such as enhanced stress tolerance. Where the promoter and its native gene are naturally occurring within the plant, i.e., in maize, transformation of the plant with these operably linked sequences also results in either a change in phenotype such as enhanced stress response or the insertion of operably linked sequences within a different region of the chromosome, thereby altering the plant's genome.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism as well as those affecting kernel size, sucrose loading, and the like.

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,885,801; 5,885,802; 5,990,389; and 5,703,049; herein incorporated by reference. Another example is lysine and/or sulfur rich seed protein encoded by the soybean 2S albumin described in U.S. Pat. No. 5,850,016 and the chymotrypsin inhibitor from barley, described in Williamson et al. (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and PCT/US97/20441, filed Oct. 31, 1997, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference). Other agronomically important genes encode latex, Floury 2, growth factors, seed storage factors, and transcription factors.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049; 5,885,801; 5,885,802; 5,990,389.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as P-Ketothiolase, PHBase (polyhydroxybutyrate synthase), and acetoacetyl-CoA reductase (see Schubert et al. (1988) J. Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content. In one embodiment, the nucleic acids of interest are targeted to the chloroplast for expression. In this manner, where the nucleic acid of interest is not directly inserted into the chloroplast, the expression cassette will additionally contain a nucleic acid encoding a transit peptide to direct the gene product of interest to the chloroplasts. Such transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Chloroplast targeting sequences are known in the art and include the chloroplast small subunit of ribulose-1,5-bisphosphate carboxylase (Rubisco) (de Castro Silva Filho et al. (1996) Plant Mol. Biol. 30:769-780; Schnell et al. (1991) J. Biol. Chem. 266(5):3335-3342); 5-(enolpyruvyl)shikimate-3-phosphate synthase (EPSPS) (Archer et al. (1.990) J. Bioenerg. Biomemb. 22(6):789-810); tryptophan synthase (Zhao et al. (1995) J. Biol. Chem. 270(11):6081-6087); plastocyanin (Lawrence et al. (1997) J. Biol. Chem. 272(33):20357-20363); chorismate synthase (Schmidt et al. (1993) J. Biol. Chem. 268(36):27447-27457); and the light harvesting chlorophyll a/b binding protein (LHBP) (Larrippa et al. (1988) J Biol. Chem. 263:14996-14999). See also Von Heijne et al. (1991) Plant Mol. Biol. Rep. 9:104-126; Clark et al. (1989) J. Biol. Chem. 264:1754417550; Della-Cioppa et al. (1987) Plant Physiol. 84:965-968; Romer et al. (1993) Biochem. Biophys. Res. Commun. 196:1414-1421; and Shah et al. (1986) Science 233:478-481.

Methods for transformation of chloroplasts are known in the art. See, for example, Svab et al. (1990) Proc. Nad. Acad. Sci. USA 87:8526-8530; Svab and Maliga (1993) Proc. Nad. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of anuclear-encoded and plastid-directed RNA polymerase. Such a system has been reported in McBride et al. (1994) Proc. Nad. Acad. Sci. USA 91:7301-7305.

The nucleic acids of interest to be targeted to the chloroplast may be optimized for expression in the chloroplast to account for differences in codon usage between the plant nucleus and this organelle. In this manner, the nucleic acids of interest may be synthesized using chloroplast-preferred codons. See, for example, U.S. Pat. No. 5,380,831, herein incorporated by reference.

The use of the term “nucleotide constructs” herein is not intended to limit the present invention to nucleotide constructs comprising DNA. Those of ordinary skill in the art will recognize that nucleotide constructs, particularly polynucleotides and oligonucleotides, comprised of ribonucleotides and combinations of ribonucleotides and deoxyribonucleotides may also be employed in the methods disclosed herein. Thus, the nucleotide constructs of the present invention encompass all nucleotide constructs that can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs of the invention also encompass all forms of nucleotide constructs including, but not limited to, single-stranded forms, double-stranded forms, hairpins, stem-and-loop structures, and the like.

Furthermore, it is recognized that the methods of the invention may employ a nucleotide construct that is capable of directing, in a transformed plant, the expression of at least one protein, or at least one RNA, such as, for example, an antisense RNA that is complementary to at least a portion of an mRNA. Typically such a nucleotide construct is comprised of a coding sequence for a protein or an RNA operably linked to 5′ and 3′ transcriptional regulatory regions. Alternatively, it is also recognized that the methods of the invention may employ a nucleotide construct that is not capable of directing, in a transformed plant, the expression of a protein or an RNA.

In addition, it is recognized that methods of the present invention do not depend on the incorporation of the entire nucleotide construct into the genome. Rather, the methods of the present invention only require that the plant or cell thereof is altered as a result of the introduction of the nucleotide construct into a cell. In one embodiment of the invention, the genome may be altered following the introduction of the nucleotide construct into a cell. or example, the nucleotide construct, or any part thereof, may incorporate into the genome of the plant. Alterations to the genome of the present invention include, but are not limited to, additions, deletions, and substitutions of nucleotides in the genome. While the methods of the present invention do not depend on additions, deletions, or substitutions of any particular number of nucleotides, it is recognized that such additions, deletions, or substitutions comprise at least one nucleotide.

The nucleotide constructs of the invention also encompass nucleotide constructs that may be employed in methods for altering or mutating a genomic nucleotide sequence in an organism, including, but not limited to, chimeric vectors, chimeric mutational vectors, chimeric repair vectors, mixed-duplex oligonucleotides, self-complementary chimeric oligonucleotides, and recombinogenic oligonucleobases. Such nucleotide constructs and methods of use, such as, for example, chimeraplasty, are known in the art. Chimeraplasty involves the use of such nucleotide constructs to introduce site-specific changes into the sequence of genomic DNA within an organism. eg, U.S. Pat. Nos. 5,565,350; 5,731,181; 5,756,325; 5,760,012; 5,795,972; and 5,871,984; all of which are herein incorporated by reference. See also, WO 98/49350, WO 99/07865, WO 99/25821, and Beetham et al. (1999) Proc. Nad. Acad. Sci. USA 96:8774-8778; herein incorporated by reference. The following examples are offered by way of illustration and not by way of limitation.

### EXAMPLES

Galactinol synthase (GOLS) and raffinose synthase (RAFS) work in concert to produce raffinose, a galactosyl-sucrose trisaccharide that accumulates during maize (Zea mays L.) seed maturation. Three cDNAs for both ZmGOLS and ZmRAFS were identified according to the invention. The ZmGOLS3 gene was transcribed late during seed development and its mRNA was present in mature seeds but declined quickly to undetectable amounts following imbibition. In contrast, ZmRAFS1 mRNA was present during seed maturation and desiccation and increased in abundance after imbibition. When seed germination was interrupted by heat, cold, salt or desiccation, ZmRAFS1 was induced by all stresses, while ZmGOLS2 was induced only by desiccation. In heat- or dehydration-stressed maize Hi-II callus cells, expression of the ZmGOLS2 gene was induced by exogenously supplied myo-inositol but not sucrose or abscisic acid (ABA). In contrast, expression of ZmRAFS1 was up-regulated by sucrose but down-regulated by raffinose and/or galactose. Callus cultured on medium containing raffinose grew better following dehydration stress than callus cultured on the same medium without raffinose. This is direct evidence that raffinose is capable of ameliorating dehydration stress in plant cells.

The raffinose family oligosaccharides (RFOs) are a group of soluble galactosyl-sucrose carbohydrates thought to play a number of roles in plant development. While some plants transport RFOs in the phloem, their accumulation in other plants is often associated with stressful environmental conditions, including cold, heat or dehydration (Santarius, 1973; Santarius and Milde, 1977; Hinesley et al., 1992; Ashworth et al., 1993; Wiemken and Ineichen, 1993; Bachmann et al., 1994). In addition, RFOs are present in many types of seeds (Amuti and Pollard, 1977). In seeds, RFOs have been ascribed three hypothetical roles: 1) contributing to desiccation tolerance (Black et al., 1996; Corbineau et al., 2000; Bailly et al., 2001); 2) increasing the longevity of seeds in the dry state (Koster, 1991; Sun and Leopold, 1993; Sun et al., 1994; Bemal-Lugo and Leopold, 1998; Buitink et al., 1998); and 3) providing a source of rapidly metabolizable carbohydrate for the first stages of germination (Main et al., 1983; Kuo et al., 1988; Nichols et al., 1993; Buckeridge and Dietrich, 1996; Dirk et al., 1999; Downie and Bewley, 2000).

The role of RFOs in stress tolerance, particularly regarding desiccation tolerance and longevity in the dehydrated state, has been intensively studied but remains controversial. RFOs are often abundant in desiccation-tolerant (“orthodox”) seeds and low or absent in desiccation-intolerant (“recalcitrant”) seeds and in seeds of ABA-insensitive mutants that cannot survive desiccation (Ooms et al., 1993; Lin and Huang, 1994; Sun et al., 1994). Additionally, RFO accumulation is often positively correlated with the onset of desiccation tolerance during orthodox seed development (Koster and Leopold, 1988; Leprince et al., 1993; Bewley and Black, 1994; Horbowicz and Obendorf, 1994; Black et al., 1996; Brenac et al., 1997a, b). Conversely, Ooms et al. (1994) have demonstrated that seeds can be germinated to the extent that they metabolize most of their RFOs but subsequently survive desiccation, indicating that large amounts of RFOs are not essential for desiccation tolerance (Hoekstra et al., 1994). Nonetheless, mature Arabidopsis seeds from aba insensitive 3 mutant plants containing progressively more severe alleles of the lesion accumulate correspondingly less RFOs and exhibit shorter survival in the dry state (Ooms et al., 1993). This is consistent with the second proposed role of RFOs in enhancing seed longevity in storage (Bemal-Lugo and Leopold, 1992; Horbowicz and Obendorf, 1994; Lin and Huang, 1994; Bernal-Lugo and Leopold, 1995). On the other hand, the importance of RFOs in enhancing seed longevity and maintaining the glassy state in dry seeds has been questioned (Bentsink et al., 2000; Buitink et al., 2000; Gurusinghe and Bradford, 2001), potentially leaving RFOs without a known role in seeds other than as an energy source (Downie and Bewley, 2000).

RFOs are synthesized by donation of galactose from galactinol to sucrose catalyzed by raffinose synthase, creating the trisaccharide raffinose. Galactinol is formed from UDP-galactose and myo-inositol by the action of galactinol synthase, which is the committed enzyme step in the RFO biosynthetic pathway (Pridham and Hassid, 1965; Lehle and Tanner, 1972; Saravitz et al., 1987; Smith et al., 1991; Hitz et al., 2002). The only known function for galactinol is in the formation of RFOs (Saravitz et al., 1987; Liu et al., 1995). Liu et al. (1998) isolated a GOLS transcript from kidney bean (Phaseolus vulgaris) seeds that increased in abundance in vegetative tissues when plants were exposed to cold stress (Liu et al., 1998). Takahashi et al. (1994) found that GOLS gene expression in rice seedlings was upregulated by chilling at 4° C. and by osmotic stress, but not by exogenously supplied ABA. Sprenger and Keller (2000) demonstrated that two different GOLS genes, both of which were upregulated by chilling, were transcribed in discrete locations (GOLS1 in mesophyll cells and GOLS2 in companion cells of the phloem) in the vegetative portions of the RFO-translocating and frost-hardy species Adjuga reptans. Transcription of a RAFS gene (AtRAFS4) was dark-induced and strongly and rapidly up-regulated by sucrose starvation in Arabidopsis leaves (Fujiki et al., 2001) and suspension-cultured cells (Fujiki et al., 2000). Anderson and Kohom (2001) demonstrated that the aerial component of Arabidopsis plants deficient in AtRAFS5 had reduced amounts of verbascose and sucrose but not of raffinose. These Atrafs5 mutants exhibited increased drought tolerance, which was attributed to altered relative carbohydrate abundance. Transgenic Arabidopsis plants overexpressing AtGOLS2 were also perturbed in normal carbohydrate amounts and exhibited greater drought tolerance through hypersensitive stomatal regulation of transpiration (Taji et al., 2002).

Despite information on RFO amounts in seeds and seed parts during development and germination, there are no data on expression of GOLS or RAFS genes in seeds during germination, or upon imbibition under stressful conditions (Peterbauer and Richter, 2001). We have characterized the expression of three GOLS and three RAFS genes in maize seeds, which accumulate substantial amounts of raffinose just prior to maturation desiccation (Bemal-Lugo and Leopold, 1992; 1995; Brenac et al., 1997a, b). As in other species, raffinose accumulation is positively correlated with the attainment of maize seed desiccation tolerance (Brenac et al., 1997a, b) and with seed longevity in dry storage (Bemal-Lugo and Leopold, 1992). In addition to studies on maize seeds, aspects of the biochemical regulation of transcription of GOLS and RAFS genes were examined in maize Hi-II callus cells. Further, we provide evidence that raffinose is capable of ameliorating dehydration stress in plant cells.

Identification and Sequence Characterization of Maize GOLS and RAFS Genes

Three GOLS cDNAs were identified from a homology search of a maize expressed sequence database using a tomato GOLS (AF311943, Downie et al., 2002) as a query. Full-length cDNAs were obtained and sequenced. These cDNAs (ZmGOLS1, AF497507; ZmGOLS2, AF497508; and ZmGOLS3, AF497509) share 78 percent identity and 90 percent similarity at the protein level. ZmGOLS2 and 3, the two most similar cDNAs, shared 93% identity at the nucleotide level. All three maize deduced amino acid sequences were most similar to the rice Water Stress Induced protein #76 (WSI76, BAA05538) (Takahashi et al., 1994) sharing 75, 70, and 76 percent identity and 89, 82, and 86% similarity for ZmGOLS1, 2, and 3, respectively.

Three RAFS cDNAs (ZmRAFS1, AF4975 10; ZmRAFS2, AF497511, and ZmRAFS3, AF497512) were identified in a maize expressed sequence database using a cucumber (Cucumis sativus) RAFS sequence (AF073744; Oosumi, C., Nozaki, J. and Kida, T. unpublished data) as a query. A full-length cDNA was isolated for ZmRAFS3, SEQ ID NO: 11 while the ZmRAFS1 SEQ ID NO: 7 and ZmRAFS2 SEQ ID NO:9 cDNAs were 3′ truncated. The cDNAs were 39% identical for the first 1590 nucleotides including the 5′ untranslated region and shared 40% amino acid sequence identity and 67% similarity over 419 N-terminal amino acids. The ZmRAFS1 deduced amino acid sequence was most similar to the RAFS from Hordeum vulgare (AAA32975. 1; Heck, G. R., Dorsett, C. and Ho, T.-H. D. unpublished data), sharing 75 percent identity and 88 percent similarity over 689 amino acids at the amino terminus deduced from the 3′ truncated maize cDNA. The ZmRAFS2 deduced amino acid sequence was most similar to the RAFS from Oryza sativa (BAB64768; Sasaki, T., Matsumoto, T. and Yamamoto, K., unpublished data) sharing 84% identity and 96% similarity over 388 amino acids deduced from the 3′ truncated maize cDNA. The full-length ZmRAFS3 SEQ ID NO: 12 deduced amino acid sequence was most similar to the RAFS from Brassica oleracea (protein id CAA55893.1, Fujikura, Y. and Karssen, C. K., unpublished data) sharing 58% identity and 85% similarity.

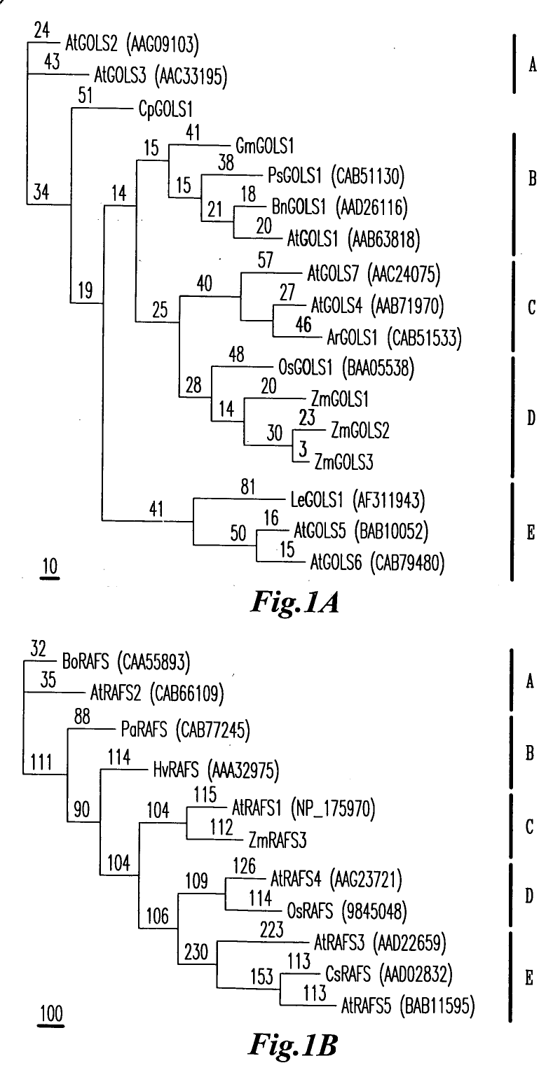

The phylogenetic relationships among the known GOLS protein sequences defined five distinct clades (FIG. 1A). Clade A was a divergent cluster of two Arabidopsis proteins that were upregulated in drought- and cold-stressed tissues (Taji et al., 2002). Along with tomato (Clade E), these two proteins had a conservative substitution of alanine for the serine identified by Sprenger and Keller (2000) as a putative phosphorylation site. All other GOLS proteins contained the conserved serine. Clade B contained GOLS from the Leguminosae and Brassicaceae and an Arabidopsis protein, the gene for which was upregulated by drought- and cold-stress (Taji et al., 2002). Clade C contained the Adjuga reptans GOLS whose gene was induced by cold in mesophyll cells (Sprenger and Keller, 2000) and two Arabidopsis proteins. Clade D contained only cereal proteins from rice and maize. Clade E contained the GOLS from tomato expressed in both seeds and leaves (Downie et al., 2002), along with two Arabidopsis proteins. The GOLS from Curcurbita pepo, a plant that transports stachyose, was orphaned, which is dissimilar to the tree proposed by Liu et al. (1998) based on more limited data.

Four of the five clades defined among known RAFS amino acid sequences had at least one member from Arabidopsis (FIG. 1B). Cereal RAFS were present in clades B, C, and D. AtRAFS4, the gene for which is induced during sucrose starvation (Fujiki et al., 2001), was present in clade D. AtRAFS5, a mutant of which was resistant to drought stress (Anderson and Kohorn, 2001), was in clade E. The remarkable similarity among GOLS deduced amino acid sequences produced the relatively short arms of the GOLS phylogenetic tree (FIG. 1A), compared to those for the more divergent raffinose synthases (FIG. 1B).

All GOLS and RAFS proteins were assessed for signal peptides using TargetP (Emanuelsson et al., 2000). There was no evidence of protein targeting to any organelle or to the apoplast.

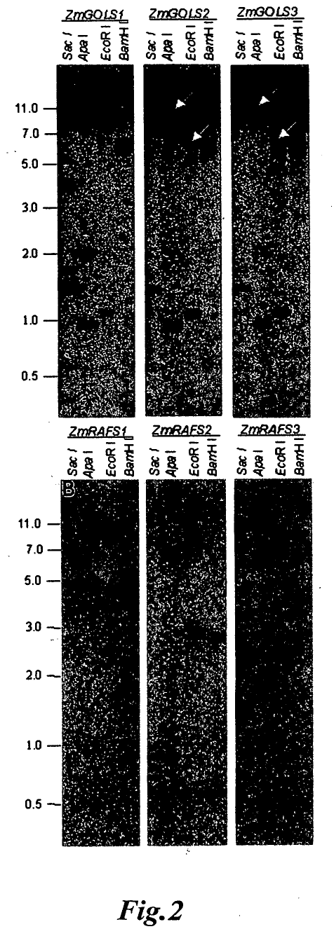

The three ZmGOLS cDNAs cross-hybridized with almost all restriction fragments from the three genes on Southern blots regardless of how stringently the membranes were washed (FIG. 2A). Sac I and Apa I ZmGOLS1-specific fragments were identified (below the asterisk, FIG. 2A) as were fragments detected only when membranes were hybridized with ZmGOLS2 or 3 (arrows, FIG. 2A) that were not distinguishable from each other. Washing blots probed with ZmGOLS2 or 3 at low stringency revealed several hybridizing restriction fragments that were no longer detectable following high stringency washes (data not shown), potentially signifying that the ZmGOLS gene family contains more than the three members reported here.

ZmRAFS cDNAs used as probes on Southern blots exhibited unique restriction fragment patterns for each gene (FIG. 2B) even when washed at low stringency (data not shown).

GOLS and RAFS Gene Expression in Maize Seeds

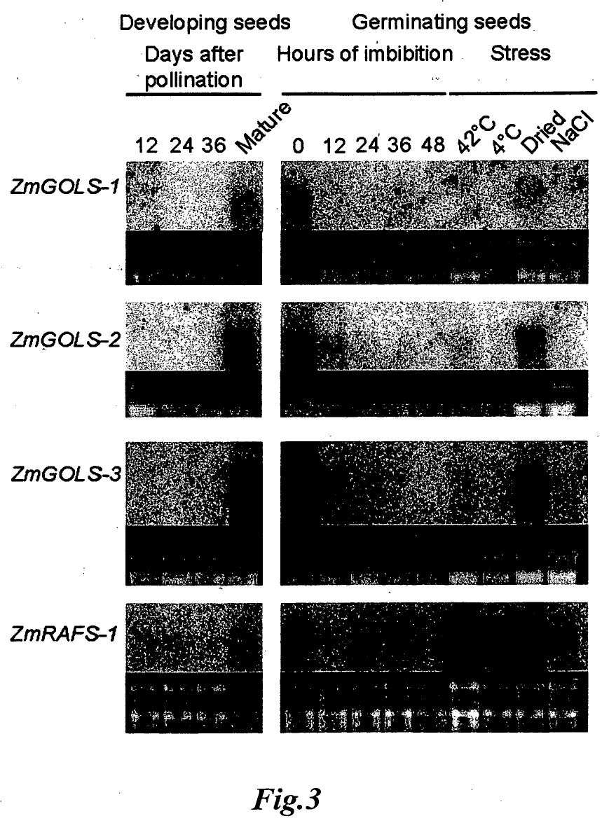

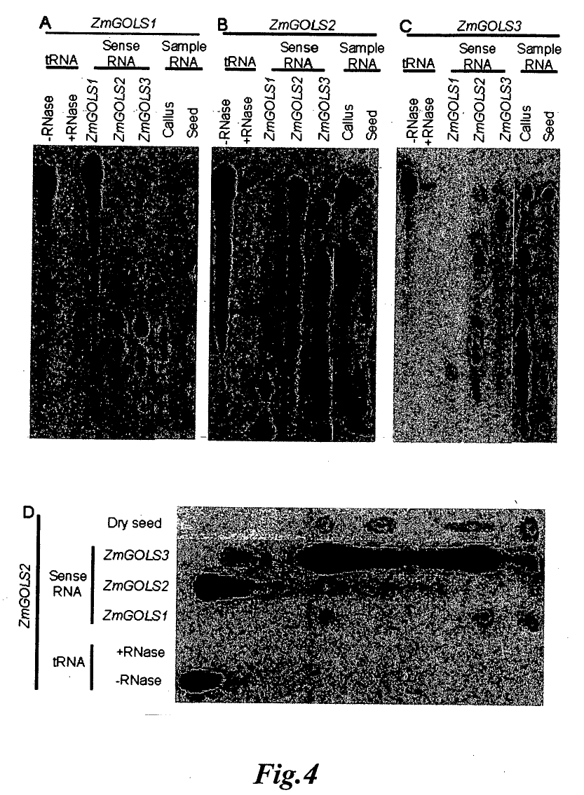

ZmGOLS mRNAs were not detectable prior to late seed maturation (between 36 and 50 days after pollination; FIG. 3). The mRNAs were present in the mature desiccated seed but were quickly degraded upon imbibition, declining to undetectable amounts by 12 h after imbibition (HAI) at 25° C. Salt-, cold-, or heat-stress failed to induce ZmGOLS gene expression while dehydration applied 24 HAI and lasting 24 h resulted in detectable quantities of ZmGOLS mRNA (FIG. 3). Once northern analysis had indicated which treatments/time points had detectable signal, ribonuclease protection assays (RPAs) were used to distinguish among the three genes potentially producing the transcripts. Each antisense RNA probe protected different portions of the three in vitro-transcribed, full-length sense RNAs, resulting in a cDNA-specific banding pattern for all three probes (FIG. 4A-D). When RNA from 24 h imbibed seeds exposed to 24 h dehydration stress was tested, no transcripts were present to protect the ZmGOLS1 probe (FIG. 4A). The ZmGOLS2 probe was protected by transcript from these seeds (FIG. 4B) while no transcript was present to afford protection to the ZmGOLS3 probe (FIG. 4C, protected band is of a size similar to that from ZmGOLS2). Hence, only ZmGOLS2 was expressed in mature, dry seeds stressed by desiccation during seed imbibition (FIG. 4B). RNA from mature, dry seeds that had not been previously imbibed was hybridized with ZmGOLS2 RPA probe but no transcript was present in the RNA to protect the probe. However, major degradation products, distinctive for fragments of ZmGOLS2 antisense probe protected by homologous regions of ZmGOLS3 sense RNA, were detected (FIG. 4D).

The amount of ZmRFS1 transcript was low in developing seeds throughout maturation and in mature, dry seeds (FIG. 3). Transcript amounts initially decreased when seeds were placed on water but then increased until 36 h (prompting the name “seed imbibition protein” or SIP by Fujikura and Karssen, Genbank submission, Accession #X79330). All stresses imposed on seeds during germination effectively increased ZmRAFS1 transcription, particularly heat shock (42° C.) and dehydration (FIG. 3). No expression of ZmRAFS2 or 3 was detected in developing seeds at any stage tested or in unstressed or stressed mature maize seeds.

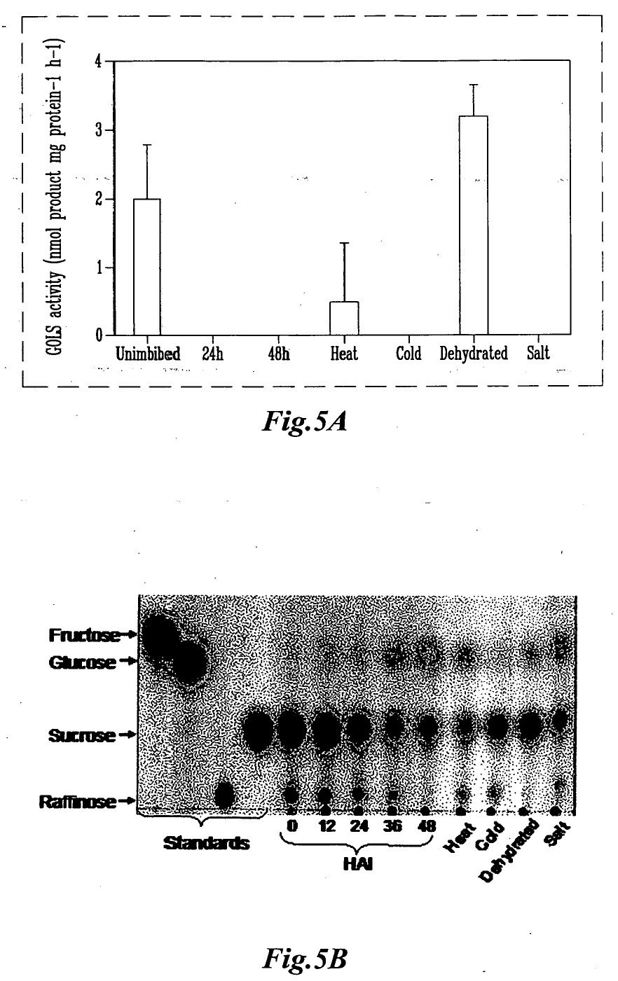

ZmGOLS enzyme activity was greatest whenever ZmGOLS transcript was detectable, e.g. in unimbibed, mature seeds (ZmGOLS3) and in seeds dehydrated after initial imbibition (ZmGOLS2) (FIGS. 3 and 5A). However, ZmGOLS2 expression and enzyme activity were not well correlated with the presence of raffinose in the seeds, as dehydration induced ZmGOLS activity but not raffinose accumulation (FIG. 5B).

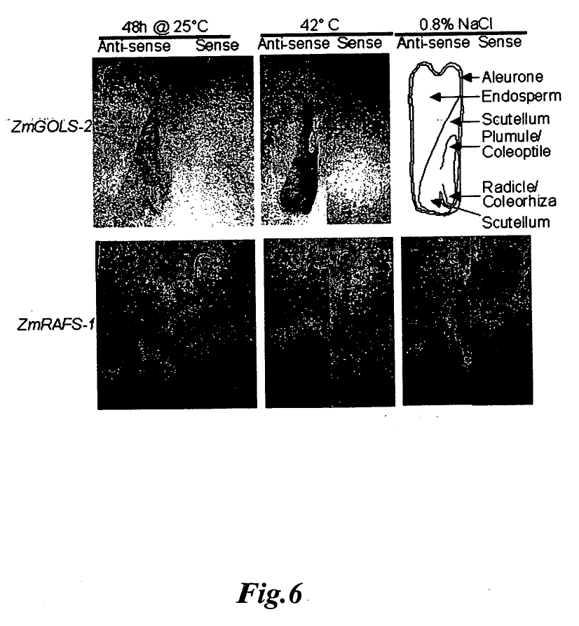

Tissue printing was used to localize the expression of ZmGOLS2 and ZmRAFS1 genes in maize seeds (FIG. 6). ZmGOLS2 mRNA abundance increased throughout the embryo (scutellum and embryonic axis) when 24 h-imbibed seeds were subjected to heat-stress for an additional 24 h compared to control seeds imbibed for 48 h (FIG. 6). ZmGOLS1 mRNA could not be detected in tissue prints while prints hybridized with ZmGOLS3 antisense riboprobes exhibited the same pattern as ZmGOLS2 (not shown). The high degree of sequence similarity between ZmGOLS2 and 3 suggested that the ZmGOLS3 probe cross-hybridized to the ZmGOLS2 mRNA, as confirmed by RPA analysis (FIG. 4). Tissue prints with dehydration-stressed seeds were inconclusive due to poor transfer of RNA from the dry tissues to the membranes.

ZmRAFS1 gene expression occurred predominately in the scutellum 48 HAI and during salt stress (FIG. 6). Upon heat shock of previously imbibed seeds, a treatment that markedly upregulated ZmRAFS1 gene expression (FIG. 3), the preponderance of the transcript was localized in the embryo (plumule, coleoptile, coleorhiza, radicle; FIG. 6). ZmRAFS2 and 3 mRNAs were not detected in seed tissue prints, consistent with results from northern blots that they are not expressed in seeds. No expression of these genes was detected in the cells comprising the aleurone layer, except in the case of ZmGOLS2, following heat shock (FIG. 6).

Raffinose Protects Callus Cells During Dehydration Stress

When Hi-II maize callus cells were grown on N6 media containing sucrose at either 2 or 5% w/v [sucrose concentrations used to maintain or induce embryos from maize callus, respectively (Armstrong and Green, 1985; Wang et al., 1995)], the final callus mass was significantly less at the higher sucrose concentration (Table 1), presumably due to osmotic stress. Inclusion of 0.1% raffinose in the medium with 5% sucrose prevented the detrimental effect of high sucrose on callus mass accumulation (Table 1). Even more striking was the marked positive effect of raffinose on the maintenance of callus growth following dehydration stress. Callus growing on either 2 or 5% sucrose was significantly and detrimentally affected by dehydration while callus growing on 5% sucrose with added raffinose grew as well as callus that had not been subjected to dehydration (Table 1). This effect was not due to an increased ability of callus supplied with raffinose to retain water during dehydration (Table 1). Despite the positive influence of raffinose on dehydration tolerance of the callus cells, raffinose was not detectable in the cells under any condition.

Table 1. Growth of Hi-II maize cells in culture on media containing sucrose and raffinose. Hi-II cells were weighed, cultured on different media for 4 d, transferred to N6 medium, cultured for another 7 d, and re-weighed. For dehydration stress, Hi-II cells were weighed, cultured on different media for 3 , dried in a flowhood for 4 h, subsampled for water content, returned to the initial media for 1 d, transferred to N6 medium, cultured for 7 days, and re-weighed. Growth rates (increase in weight relative to initial weight) and water contents (dry weight basis) are the means of three replicates. Analysis of variance and mean separations were calculated among media within a dehydration stress treatment.

| | Relative weight gain | | Water content | |
|---|---|---|---|---|
| | | Dehydration | | Dehydration |
| Medium | Control | stressed | Control | stressed |
| 2% sucrose | 1.89 ± 0.17 a | 1.35 ± 0.16 b | 95.08% ± 0.35% a | 92.22% ± 1.23% a |
| 5% sucrose | 1.45 ± 0.04 b | 1.37 ± 0.02 b | 92.29% ± 0.44% b | 87.95% ± 1.22% b |
| 5% sucrose + 0.1% | 1.80 ± 0.10 ab | 1.82 ± 0.02 a | 93.03% ± 0.32% b | 89.03% ± 0.55% b |
| raffinose | | | | |

*Growth rates and water contents followed by different letters within columns are significantly different with 95% confidence.

ZmGOLS and ZmRAFS Gene Expression in Callus Tissue

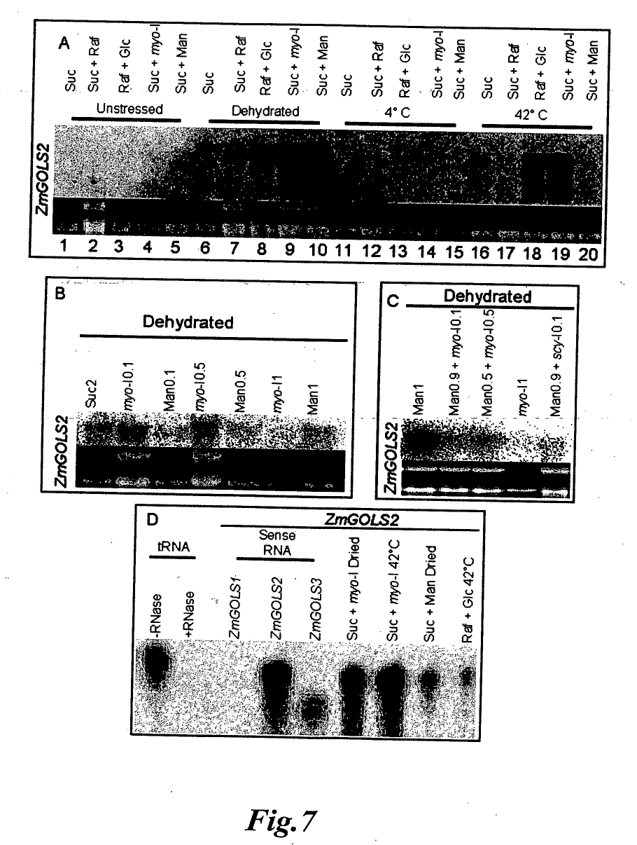

ZmGOLS2 expression was not detected in unstressed Hi-II callus, regardless of the medium composition (FIG. 7A, lanes 1-5). [Only ZmGOLS2 RNA was detected by RPA when used with heat-stressed (24h, 42° C.) callus RNA (FIG. 4B).] Although medium containing 5% sucrose resulted in a decrease in callus growth relative to 2% sucrose (Table 1), ZmGOLS2 expression was not induced (FIG. 7A, lane 1). Cold-stressed callus (4° C., 24 h) did not express ZmGOLS2 on any of the medium compositions assayed (FIG. 7A, lanes 11-15). However, both dehydration (as in Table 1; FIG. 7A, lanes 6-10) and heat stress (42° C., 24 h; FIG. 7A, lanes 16-20) upregulated ZmGOLS2 transcription. Moreover, the degree of ZmGOLS induction in stressed callus varied considerably depending on the medium composition (FIG. 7A). Dehydration or heat stress upregulated ZmGOLS2 expression only slightly in cells on 5% sucrose or on 2% each of sucrose and raffinose (FIG. 7A, lanes 6, 7, 16, and 17). However, both stresses resulted in ZmGOLS2 mRNA accumulation on media containing 2% each of raffinose and glucose or 2% sucrose plus 0.01% of the GOLS substrate myo-inositol (FIG. 7A, lanes 8, 9, 18 and 19). Callus cells on 2% sucrose and 7.8% mannitol (4.5 times the osmotic potential of 5% sucrose) also upregulated ZmGOLS2 gene expression when subjected to dehydration but not to heat stress (FIG. 7A, compare lanes 10 and 20).

To further investigate the responsiveness of ZmGOLS2 expression of dehydration-stressed callus cells to myo-inositol and mannitol, cells were grown on 2% sucrose supplemented with myo-inositol or mannitol, singly or in combination (FIGS. 7B and C). Dehydrated cells on low amounts of myo-inositol (0.01-0.1% w/v; FIGS. 7A and B, respectively) upregulated ZmGOLS2 transcription while high myo-inositol (1% w/v; FIGS. 7B and C) concentrations repressed ZmGOLS2 transcription below that on 2% sucrose alone (FIG. 7B). While very high mannitol concentrations (7.8% w/v) upregulated ZmGOLS2 transcription in dehydrated cells (FIG. 7A) low mannitol concentrations (0.1-0.5% w/v) repressed ZmGOLS2 transcription below that present in cells grown on 2% sucrose alone (FIG. 7B). However, keeping the calculated osmotic potential of the media constant and altering the concentrations of myo-inositol and mannitol, low myo-inositol (0.1%) combined with high mannitol (0.9%) did not act synergistically to increase ZmGOLS2 expression (FIG. 7C). Low concentrations of the naturally occurring myo-inositol isomer, scyllo-inositol, in combination with high mannitol did not alter the expression of ZmGOLS2 relative to similar concentrations of myo-inositol and mannitol (FIG. 7C).

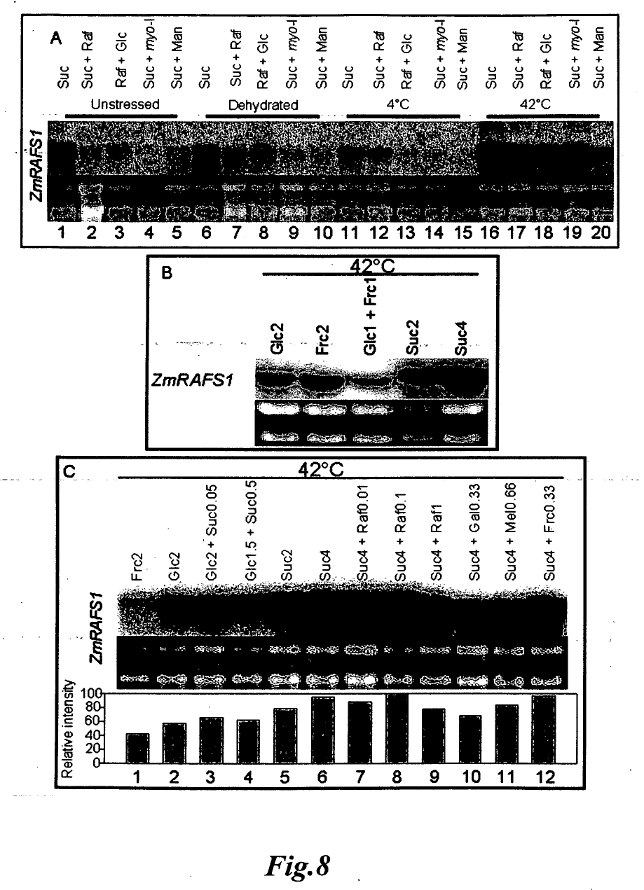

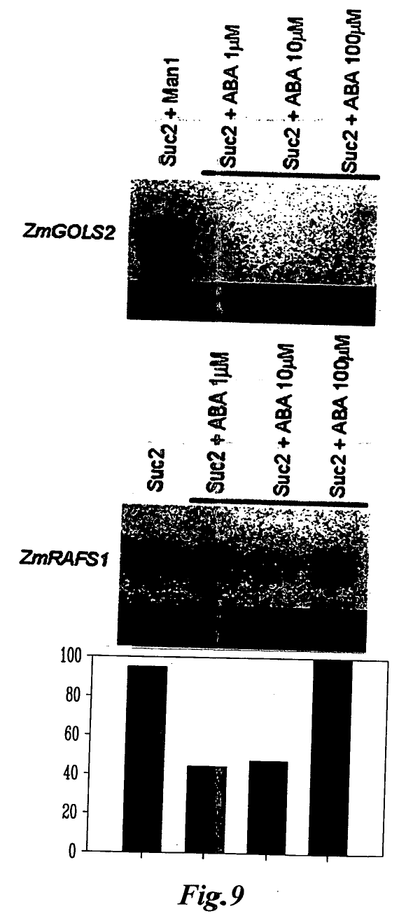

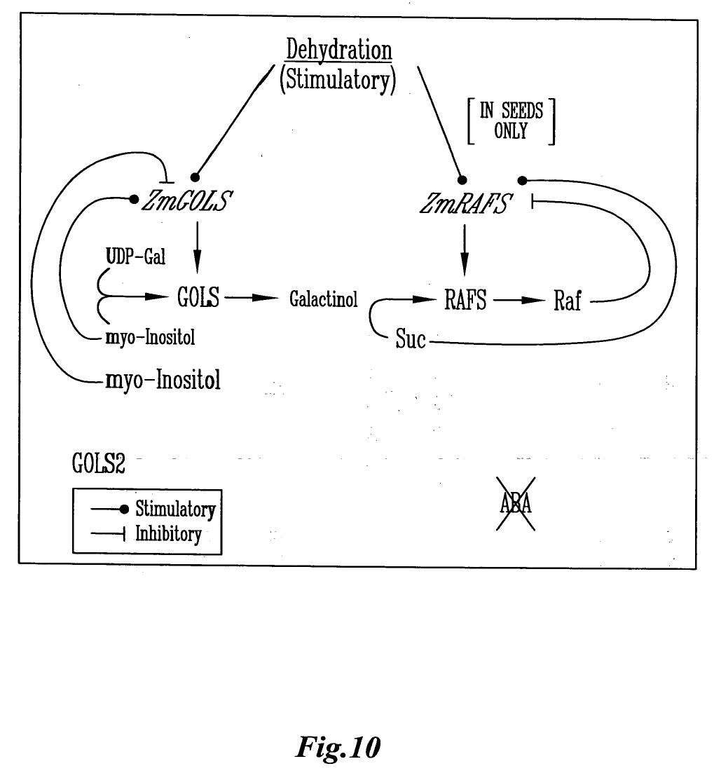

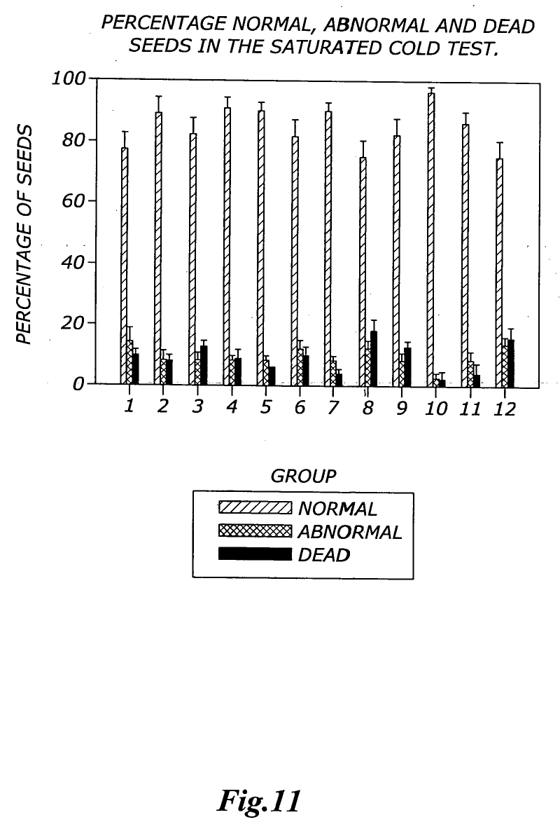

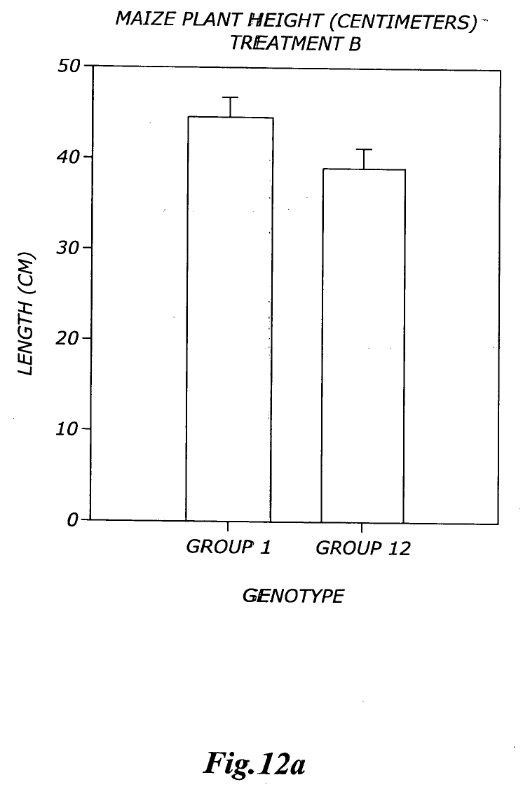

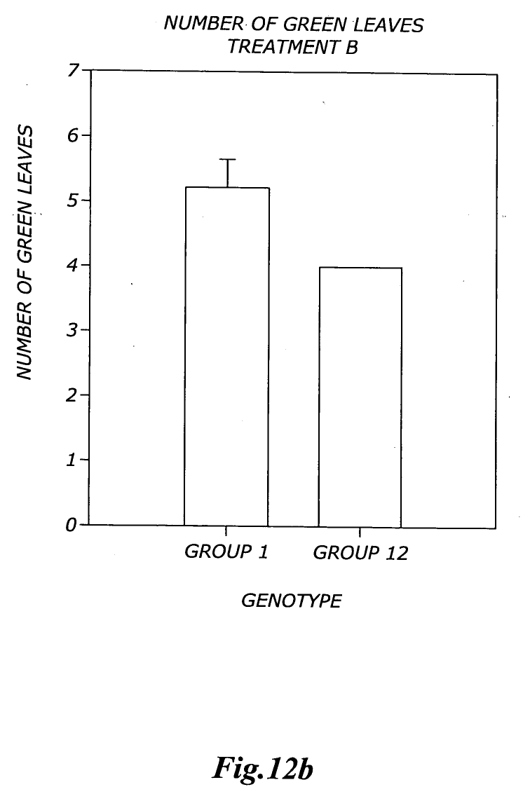

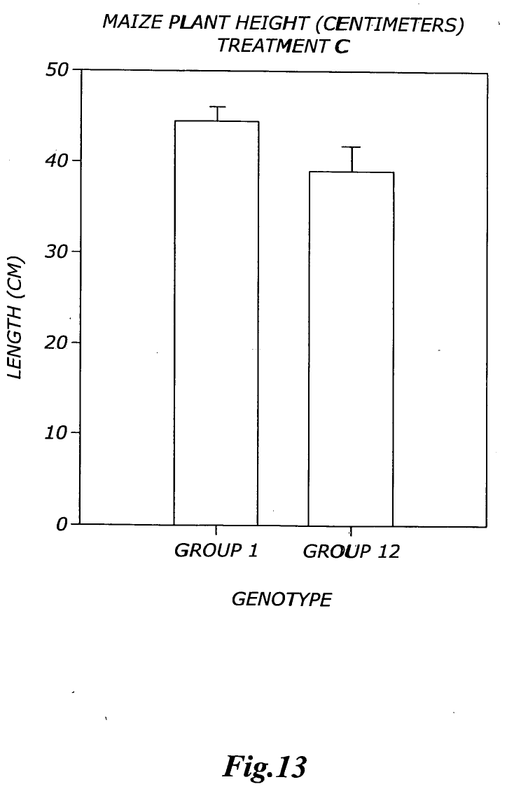

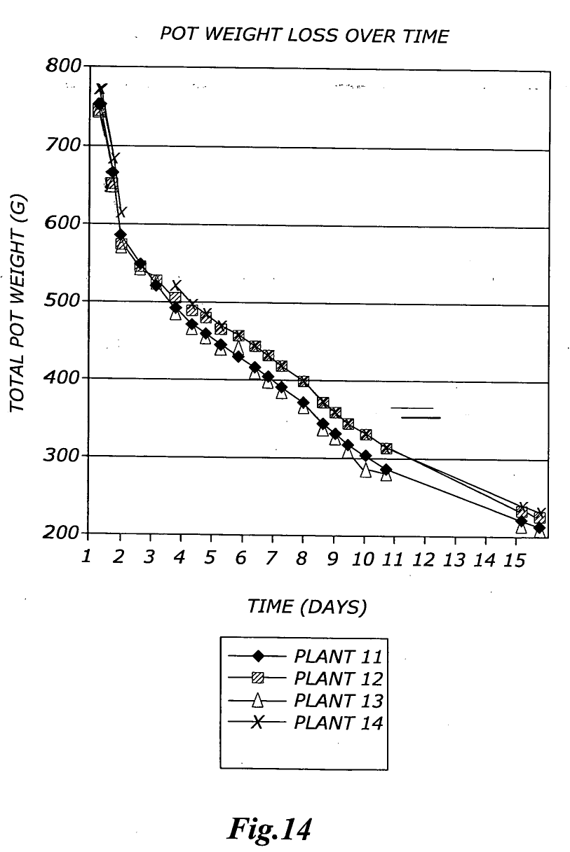

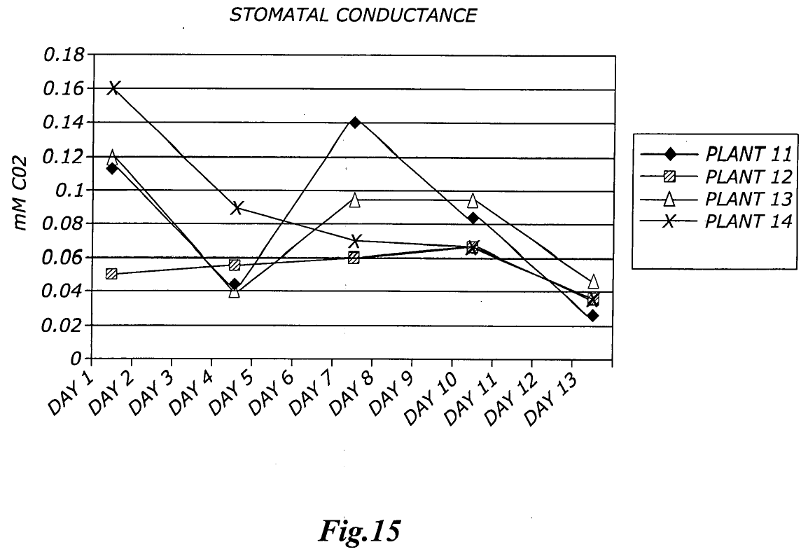

ZmRAFS1 was the only RAFS mRNA detected in callus. ZmRAFS1 was expressed on sucrose media regardless of imposed stress (FIG. 8A, lanes 1, 6, 11, and 16), although heat shock was the most effective treatment. The inclusion of myo-inositol in the media did not elicit greater ZmRAFS1 mRNA accumulation with the exception of cells exposed to heat shock (FIG. 8A, lane 19). Mannitol did not influence ZmRAFS1 gene expression (FIG. 8A, lanes 5, 10, 15, and 20).

Gene expression of ZmRAFS1 was up-regulated by sucrose in maize Hi-II callus cells (FIG. 8A, B, and C). The inclusion of 2% (w/v) or greater sucrose increased transcription of ZmRAFS1 (FIG. 8C, lane 5). This increase in ZmRAFS1 expression as sucrose concentration increased was not due to higher osmotic concentration of the media. The osmotic concentration in lanes 2-5 (FIG. 8C) either decreased, if sucrose was taken up directly, or remained the same, if sucrose was first hydrolyzed to glucose and fructose. It is unlikely that the component monosaccharides of sucrose are responsible for the induction of ZmRAFS1, since neither was capable of eliciting an increase in ZmRAFS1 expression when provided at the same or greater osmotic concentration, either singly or in combination (FIGS. 8B, 8C, lanes 1 and 2).

Raffinose may inhibit the expression of ZmRAFS1 upon attaining a sufficient concentration (FIG. 8C, compare lanes 6-8 with 9). This inhibition could be exerted by raffinose itself or by its metabolite galactose, which was as effective as raffinose at repressing ZmRAFS1 expression at equal galactose molarities (FIG. 8C, lane 10). Although no eukaryotic raffinose porters have yet been cloned, raffinose is thought to be taken up directly by intact maize cells (Heyser et al., 1976) and physiological evidence for proton-raffinose symporters in plant plasmamembranes exists in species exhibiting symplasmic minor-vein configurations (van Bel et al., 1996).

Expression of both ZmGOLS2 and ZmRAFS1 genes was consistently upregulated upon the imposition of dehydration stress, whether in seeds or callus. It is possible that the upregulation of both genes was in response to abscisic acid (ABA) accumulation elicited by stress, so we tested whether applied ABA was capable of inducing ZmGOLS2 or ZmRAFS1 gene expression in unstressed cells growing on N6+2% (w/v) sucrose. The ZmGOLS2 gene was not responsive to ABA, although it was clearly induced by 1% (w/v) mannitol and dehydration (positive control lane, FIG. 9). ZmRAFS1 gene expression was also not induced beyond that of 2% sucrose alone when ABA was supplied exogenously (FIG. 9).

Discussion

GOLS and RAFS Phylogeny

The greater degree of similarity among GOLS proteins relative to RAFS proteins was evident from the much shorter arm lengths in the phylogenetic tree for GOLS (FIG. 1). There were five clades identified for GOLS. Clade A contained two drought and cold upregulated (Taji et al., 2002) Arabidopsis proteins, AtGOLS2 and 3 that had a conserved serine to alanine substitution eliminating the serine identified by Spenger and Keller (2000) as a potential phosphorylation site. The only other GOLS to encode this amino acid substitution was tomato (AF311943) in clade E. Clade B housed the two members from the leguminosae along with Brassica and Arabidopsis AtGOLS1 (also drought and cold upregulated (Taji et al., 2002)) proteins. Clade C contained Arabidopsis AtGOLS4 and 7 and the Adjuga reptans GOLS1 that is present in the mesophyll and thought to be involved in the manufacture of storage RFO (Sprenger and Keller, 2000). Clade D was comprised of all 4 monocot GOLS proteins. Clade E contained AtGOLS5 and 6 and the tomato LeGOLS1 while the GOLS from cucumber was orphaned. The five clades identified among the RAFS deduced amino acid sequences are rather enigmatic as to their relationship. Even the RAFS proteins from the monocots are dispersed over three of the five clades.

ZmGOLS and ZmRAFS Transcription in Seeds

The last two enzymes in the raffinose biosynthetic pathway were first transcribed in maize seeds between 36 and 50 DAP (FIG. 3), consistent with the accumulation of raffinose late during maize seed maturation (Brenec et al., 1997a). The mRNAs of ZmGOLS3 and ZmRAFS1 were stored in mature, desiccated seeds and were rapidly degraded following imbibition, although ZmRAFS1 transcript began to re-accumulate at 12 HAI. RPA analysis revealed that ZmGOLS3 was synthesized in developing maize seeds and was present as a stored message in mature, dehydrated kernels but that, following imbibition, seeds were capable of synthesizing only ZmGOLS2 in response to heat or dehydration stress. There are numerous examples of differentially regulated genes that are expressed during seed development and late maturation, accumulate transcript that is stored in the mature, dehydrated seed, and subsequently degraded during imbibition and not re-synthesized during germination or, in some cases, at any time during the vegetative life cycle (Hongtrakul et al., 1998; Wohlfarth et al., 1998; Aalen, 1999; Carranco et al., 1999; Castillo et al., 2000). Additionally, proteins that promote anabolic processes during seed development, and the transcripts from which they are made, are generally degraded during germination (Adachi et al., 1993). The putative dual roles of raffinose in providing a rapidly metabolizable source of energy during germination (Kuo et al., 1988; Downie and Bewley, 2000) as well as providing protection against a variety of stresses (Liu et al., 1998; Taji et al., 2002) may be regulated in planta through differential expression of GOLS genes. Hence, in maize seeds, ZmGOLS3 is responsible for participating in raffinose production during seed development while ZmGOLS2 is responsible for participating in raffinose production to possibly ameliorate environmental stress during germination. Tissue-specific and differential expression of different GOLS genes has been documented previously in leaves of the raffinose-translocating species Adjuga reptans (Sprenger and Keller, 2000), but this is the first report of temporal partitioning of GOLS gene expression.

It required 48 h of germination on water at 25° C. for the raffinose present in maize seeds to decline to undetectable amounts (FIG. 5B), which is in agreement with other accounts (Koster and Leopold, 1988). Of the stresses imposed on 24 h germinating seeds, only 24 h dehydration resulted in a decrease in raffinose content below the level of detectability. Based on: 1) the failure of cold- or salt-stresses to induce ZmGOLS2 transcription in seeds; 2) the lack of GOLS activity in seeds treated in this manner; and 3) the presence of raffinose in unstressed, germinating maize seeds at 24- and 36-HAI, it is probable that heat-shock, chilling, or salt exposure during germination simply prevented the further metabolism of pre-existing raffinose rather than eliciting its re-accumulation. In contrast to heat-, cold-, and salt-stressed seeds, 24 h-germinated seeds exposed to dehydration were capable of metabolizing raffinose to very small amounts, as has been observed in Arabidopsis seeds (Ooms et al. 1994). This decrease occurred despite the fact that redrying previously imbibed maize seeds induced the transcription of both ZmGOLS2 and ZmRAFS1 and enhanced GOLS activity in maize seeds (FIG. 5A). If it is assumed that an active RAFS is produced from the transcript that accumulates due to dehydration (FIG. 3), it is likely that the activity of α-galactosidase outstrips the raffinose synthetic capability of the dehydrating, post-imbibition seed, preventing any net raffinose accumulation. Nonetheless, the shift in production of ZmRAFS1 transcript from the scutellum of the germinating seed to the embryonic axis of heat-stressed seeds is perhaps of physiological significance (FIG. 6). The cells of the embryonic axis have been shown to be more sensitive to stress than cotyledon or aleurone cells (Koster and Leopold, 1988; Golovina et al., 1997). We have shown that raffinose, even in very small amounts (0.1% w/v), is capable of alleviating dehydration-induced damage in desiccation-sensitive maize Hi-II callus cells (Table 1). Our findings corroborate the demonstration by Xiao and Koster (2001) that exogenously supplied raffinose/sucrose mixtures can impart dehydration tolerance to plant cells in vitro.

Environmental Control of ZmGOLS and ZmRAFS Transcription in Seeds and Callus Cells:

A greater variety of environmental perturbations influenced ZmRAFS1 expression in seeds than was the case for ZmGOLS2 expression (FIG. 3). However, the opposite was true in maize callus cells (FIGS. 7A and 8A).

ZmRAFS1 expression was upregulated in seeds by heat and dehydration stresses while only heat stress upregulated ZmRAFS1 in callus cells (FIGS. 3 and 8A). However, the discrepancy in the degree of induction of ZmRAFS1 transcription evoked by dehydration of seeds (FIG. 3) and callus (FIG. 8A) was probably due as much to a shorter 4 h and less severe (no more than ˜4% water loss; Table 1) dehydration stress in callus than in seeds (24 h dehydration resulting in a water loss of 11±0.7%, mean and standard error) as it was to tissue-specific differences in gene expression. Moreover, ABA was ineffective in inducing the transcription of ZmGOLS and ZmRAFS genes (FIG. 9 and data not shown). Because GOLS and RAFS must act in concert in the production of raffinose in stressed plant cells, requiring coordinated gene regulation. However, it is not uncommon to have stress-regulated genes that are not responsive to ABA (Gilmour and Thomashow, 1991; Ishitani et al., 1997).

Metabolite Control of ZmGOLS and ZmRAFS Transcription in Seeds and Callus Cells

Myo-inositol, a precursor of galactinol, enhanced ZmGOLS2 mRNA accumulation in dehydrated and heat-shocked callus cells (FIG. 7A and B). ZmGOLS2 mRNA declined to undetectable amounts during germination, despite increasing free myo-inositol availability in the seed at this time (Henry, 1976). This is consistent with upregulation of ZmGOLS2 transcription by myo-inositol only in dehydrating and heat-shocked cells and then only at low myo-inositol concentrations (FIGS. 7A and B). Soybean seeds deficient in myo-inositol-1-phosphate synthase failed to accumulate normal RFO amounts during development (Hitz et al., 2002). In mutant seeds, exogenously supplied myo-inositol resulted in greater accumulations of galactinol and RFOs than in seeds without added myo-inositol. However, exogenous myo-inositol also increased the amounts of RFOs in wild type seeds, suggesting that the supply of myo-inositol is a controlling factor in RFO accumulation during seed development (Hitz et al., 2002). Whether this enhancement of RFO synthetic ability was via gene upregulation and/or by greater substrate availability is not currently known.

Materials and Methods

Phylogenetic Analysis of GOLS and RAFS Proteins From Dicots and Monocots

All known full-length deduced amino acid sequences for GOLS and RAFS enzymes were obtained from Genbank and aligned with the full-length, deduced amino acid sequences for the maize homologs using ClustalW multiple-sequence alignment software (Thompson et al., 1994). For both GOLS and RAFS, the Clustal aligned proteins were arranged in a phylogenetic tree using a beta version of PAUP v4.0b10 (Swofford, 1998). Since all full-length forms of both GOLS and RAFS identified to date have come from angiosperms, unrooted trees were constructed.

The deduced amino acid sequences for the GOLS proteins used in the analysis included one from each of: Lycopersicon esculentum (Accession AF311943); Adjuga reptans (CAB51533); Brassica napus (AAD26116); Glycine max (Kerr et al., 1997); Pisum sativum (CAB51130); Cucurbita pepo (Kerr et al., 1997); Oryza sativa (BAA05538, Takahashi et al. 1994); 7 from Arabidopsis thaliana numbered according to Taji et al. (2002) (BAB10052, Sato et al., 1997), (AAB63818 and AAG09103, Lin et al., 1999) (AAB71970, AAC33195, AAC24075 and CAB79480) and 3 from Zea mays.

The deduced amino acid sequences used to construct the tree for RAFS included one each from Brassica oleracea (CAA55893), Persea americana (CAB77245), Cucumis sativus (AAD02832), Oryza sativa (sequence annotated from 9845048), Hordeum vulgare (AAA32975), Zea mays and five from Arabidopsis numbered in ascending chromosomal residence and location, (AtRAFS1, NP_{—}175970, At1g55740; AtRAFS2, NP_{—}191311, At3g57520; AtRAFS3, NP_{—}192106, At4g01970; AtRAFS4, NP_{—}197525, At5g20250; AtRAFS5, NP_{—}198855, At5g40390).

The Clustal-aligned sequences were analyzed in a heuristic search using random stepwise addition of taxa and replicated 100 times to converge on the most optimal tree. To extend the search beyond local optima on which stepwise addition is prone to converge (Swofford, 1998), the number of trees held for re-evaluation upon adding the next taxon was set to five throughout tree construction (Swofford, 1998). To further guard against defining a phylogenetic tree of local optima a shallow descent, tree bisection and reconnection branch swapping algorithm was invoked according to the recommendations of Swofford (1998) once all 18 GOLS and 11 RAFS entries had been added. The clades defined in the heuristic search were verified using 100 bootstrapped replications in a second heuristic search. The branch lengths assigned to each node in the heuristic search were used in the tree produced from the PAUP results by TREEVIEW (Page, 1996).

Plant Material

Mature maize (Zea mays L.) seeds from inbred line plants. Seeds of different developmental stages were recovered from tagged ears harvested at 12, 24, 36, and 50 days after controlled pollination.

Reagents

Media and chemicals were purchased from Sigma (St. Louis, Mo., USA) unless otherwise noted. Restriction enzymes were from New England Biolabs (NEB, Inc., Beverly, Mass., USA) and radioisotopes were from New England Nuclear (NEN Life Science Products, Inc., Boston, Mass., USA) unless otherwise noted.

Germination Conditions and Treatments

Distilled, deionized water (15 mL) saturated two 8.5-cm diameter blotting paper disks (Grade 628, Stults Scientific Eng., Springfield, Ill., USA) in a Petri dish (Fisher Scientific, Springfield, N.J., USA). Maize seeds (10 g) were rinsed with 70% ethanol, washed in distilled water, and sown on the blotters. Dishes were placed at 25° C. in the dark. Some seeds, initially sown on water at 25° C., were subsequently transferred to colder (4° C.) or warmer (42° C.) germination temperatures, moved to dishes containing blotting papers saturated with salt solution (138 mM NaCl), or removed from water (dried at 25° C. for 24 h to 6% moisture content [fresh weight basis]).

Hi-II Cell Proliferation, Media Composition, Stress Imposition and Growth Rate Evaluation

Maize Hi-II callus cultures were initiated according to Armstrong and Green (1985) from immature embryos isolated from Hi-II maize plants provided by Pioneer. Cultures were incubated in the dark at 25° C. on solid N6 medium containing 2% (w/v) sucrose (Chu et al., 1975) amended with 2 mg/L 2,4-dichlorophenoxyacetic acid, 25 mmol/L L-proline, and 10 μmol/L AgNO₃, pH 5.8 (N6S2 medium) and sub-cultured every two weeks onto fresh media. For experimental treatments, approximately 2 g of cells (fresh weight) were added to each 100×15 mm plate. To determine the effect of various sugars and environmental stresses on the expression of ZmGOLS and ZmRAFS genes in maize Hi-II callus cells, callus was cultured on N6 media supplemented with different concentrations of sugars and: a) grown at 25° C. in the dark (control conditions); b) exposed to 42° C. for 24 h (heated); c) exposed to 4° C. for 24 h (chilled); or d) exposed to a current of air at 25° C. in a laminar flow hood for 4 h (dehydrated). To determine the responsiveness of ZmGOLS and ZmRAFS genes to exogenous ABA, calli were grown on media with 1, 10, and 100 μM cis, trans-ABA (Sigma) under control conditions for 1 d prior to harvest and RNA extraction.

Effect of Galactinol and Raffinose Precursors/Products on ZmGOLS and ZmRAFS Gene Expression

Sucrose and raffinose amounts in N6 media were altered, singly for sucrose or in combination with raffinose, to determine if RAFS substrates or the final product of the coordinate action of GOLS and RAFS influenced ZmGOLS and ZmRAFS gene expression. Because it is not known whether sucrose or raffinose are taken up into Hi-II cells directly or if they are hydrolyzed prior to uptake, glucose, fructose, galactose, and melibiose were also supplied. Myo-inositol (1,2,3,5-trans-4,6-hexahydroxy cyclohexane, Sigma), a GOLS substrate, was added to N6S2 media at amounts from 0.01-1% w/v. To determine if gene expression was regulated by osmotic stress, N6S2 media was amended with 0.1% to 7.8% w/v mannitol. Callus cells were also grown on N6 with combinations of sugars and the myo-inositol isomer, scyllo-inositol. All chemicals were sterilized by filtration and added to autoclaved media cooled to 65° C. just prior to pouring the plates. Hi-II callus cells on these various media were maintained under control conditions or subjected to stress (see above) prior to being harvested for sugar or RNA extraction or moisture content analysis. Cells were transferred to amended N6 media for 8 days, the environmental stress (if any) applied, and the cells harvested.

Effect of Raffinose in Protecting Hi-II Callus During Dehydration Stress

Samples of Hi-II cells growing on N6S2 were weighed and placed on N6S2, N6S5, or N6S5 supplemented with 0.1% w/v raffinose. Callus was grown for 4 d at 25° C. on these various media. The callus on a particular medium was separated into two equal sub-samples and the fresh weight obtained. A sub-sample of half of the callus cells per plate for each medium was subjected to dehydration stress by removal from the medium and exposure to a current of air in a flow hood for 4 h. The remaining half of the callus cells were not exposed to stress. After 4 h dehydration stress the callus was placed back on its respective medium for an additional 22 h. Previously stressed and non-stressed calli were then transferred to N6S2 for one week. At the end of the week the calli were weighed again to determine how much growth had occurred during the week. The growth of the cells was expressed as the increase in fresh weight after one week of growth on N6S2 relative to the callus fresh weight 7 days earlier (Dennehey et al.; 1994). This experiment was repeated 3 times.

The water content of the cells that had and had not been subjected to dehydration was determined immediately following the 4 h dehydration treatment. Sub-samples of the stressed and unstressed cells also were extracted to determine the identity and quantity sugars in the cells at this time (see below). Cells that had or had not been dehydrated were weighed and then dried at 105° C. for 16 h. Cells were then cooled over activated alumina (Grabe, 1989) and the dry weight recorded.

DNA Sequencing

Sequencing was performed at the Macromolecular Structure Analysis Laboratory (University of Kentucky, Lexington, Ky., USA). An ABI Prism 377 DNA Sequencer (ABI; Perkin-Elmer, Foster City, Calif., USA) and dye termination chemistry with AmpliTaq DNA polymerase, FS (Taq; FS; Perkin-Elmer/Applied Biosystems Division [PE/ABI], Foster City, Calif., USA) was used to read cycle-sequencing reactions employing a combination of universal and gene-specific primers (Operon Technologies, Alameda, Calif., USA; Integrated DNA Technologies, Inc., Coralville, Iowa, USA).

Genomic DNA Isolation and Analysis

Genomic DNA was isolated from expanding maize leaves according to Murray and Thompson (1980). Genomic DNA (5 μg per lane) was exhaustively digested with restriction endonucleases, electrophoresed through a 0.8% agarose gel in 1×TAE (Sambrook, 1989) and transferred to GeneScreen membrane (New England Nuclear (NEN) Life Science Products, Inc., Boston, Mass., USA). The digested DNA was cross-linked to the membrane and prehybridized at 65° C. for 12 h in 6×SSPE, 5× Denhardt's solution (Denhardt, 1966), 0.5% SDS, and 100 μg ml⁻¹ boiled, sheared salmon sperm DNA. Thereafter, the blots were hybridized with randomly-labeled (Feinburg and Vogelstein, 1983) radioactive ([³²P] dCTP) cDNAs. Membranes were first washed twice, 15 min each time, at low stringency (2×SSC, 0.1% SDS at 65° C.) and exposed to a phosphor screen for 2 days. The image was captured using a Phosphorlmager 445 SI (Molecular Dynamics, Sunnyvale, Calif., USA) and the blots re-washed at high stringency (0.2×SSC, 0.1% SDS, 65° C. 1×30 min) and re-exposed to the phosphor screen for 5 days.

RNA Isolation and Analysis

Total RNA from seeds, regardless of treatment, was extracted according to Wan and Wilkins (1994). Total RNA (20 μg per lane) from seeds was transferred onto positively charged nylon membranes (Amersham Life Science Inc., Arlington Heights, Ill., USA) in 10×SSC (1×SSC is 150 mM NaCl and 15 mM Na citrate pH 7) overnight, the blot dried and UV cross-linked at 50 mJoules on a GS genelinker UV chamber (Bio-Rad Laboratories, Hercules, Calif., USA). After rinsing the membranes for 5 min in 2×SSC, blots were placed in pre-hybridization solution (50% formamide, 5× Denhardt's solution (Denhardt 1966), 100 μg ml⁻¹ boiled, sheared salmon sperm DNA, 0.2% SDS, 6×SSC pH 7.0, Sambrook et al. 1989) for 4-6 h at 42° C. Randomly labeled, radioactive cDNA probes ([³²P] dCTP) were hybridized to the blots for at least 12 h at the same temperature as pre-hybridization. The primary wash was done in 2×SSC, 0.1% SDS, RT for 5 min, and repeated but at 65° C. for 30 min. The two final high stringency washes were in 0.2×SSC, 0.1% SDS at 65° C. for 30 min each. The hybridized probe was detected by autoradiography using Kodak X-OMAT X-ray film (Eastman-Kodak Ltd.) or on a phosphoimaging screen using a Phosphorlmager (Molecular Dynamics).

Tissue prints of seeds imbibed on water for 48 h at either 4, 25 or 42° C., imbibed on 0.8M NaCl, or imbibed and then dehydrated were bisected longitudinally and each half pressed onto separate membranes for 30 seconds, providing two membranes of prints from the same seeds, one for the sense and one for the anti-sense probe. The two prints were UV cross-linked and one was hybridized with a digoxigenin-labeled (Roche Molecular Biochemicals, Indianapolis, Ind., USA) sense-RNA and the other with an antisense-RNA probe. Each RNA probe was prepared and quantified using serial dilutions of the probes and labeled, control RNA (Roche) in dot-blots followed by chemiluminescent detection. The two prints for each gene were prehybridized and then exposed to the same specific activity of sense or anti-sense probe, respectively, as estimated from the dot-blots. Subsequent steps were performed as described in Nonogaki et al. (2000).

Ribonuclease Protection Assay

The high degree of sequence homology among ZmGOLS genes prompted verification of results from northern blot analysis using RPAs (RPA III, Ambion, Little Rock, Tex., USA). Templates for RPA antisense probes were amplified by PCR using the respective cDNA as a template and adding a T7 promoter sequence (lower case) to the 3′ primer. Primers were designed as follows: GOLS1 5′ primer 5′>GACCACACTCAAACCAGCCA<3′, SEQ ID NO:13 GOLS1 3′primer 5′>taatacgactcactatagggCAGAAGGGCCACCAC<3′ SEQ ID NO:14, GOLS 2 and 3 5′ primer 5′>AACCGCCAGCCAACAAACAG<3′ SEQ ID NO:15, GOLS 2 and 3 3′ primer 5′>TAATACGACTCACTATAGGGTCGCCCGCCAGGAA<3′ SEQ ID NO:16. PCR conditions were 96° C. 3 min at the end of which TAQ in buffer was added to each tube for a hot start followed immediately by 36 cycles (94° C. for 30s, 58° C. for 30s, 72° C. for 1 min). PCR products were separated by agarose gel electrophoresis and purified from the gel (Qiaquick, Qiagen Valencia, Calif., USA). Plasmid (pSPORT, Life Technologies, Inc., Rockville, Md., USA) containing the full-length cDNA of each ZmGOLS was used as template for the synthesis of sense control RNA using T7 RNA polymerase following linearization with NotI, gel purification and recovery (Qiagen). ³²P-UTP labeled antisense RNA probes against the 5′ UTR region of ZmGOLS1 (277 bp probe from 33 to 309 bps), ZmGOLS2 (190 bp probe from 35 to 224 bps) and ZmGOLS3 (186 bp probe from 52 to 233 bps plus an additional 4 bps from the 5′ primer, see below) were synthesized using a kit-based, run-off transcription reaction (MAXIscript, Ambion). Full-length probes were gel purified by denaturing polyacrylamide electrophoresis, excised, eluted and quantified according to the manufacture's instructions (Ambion).

For ZmGOLS1 3.0×10⁵ CPM probe was used per reaction, ZmGOLS2 probe was used at 1.3×10⁴ CPM, while ZmGOLS3 probe was added at 2.1×10⁴ CPM per reaction. Probes were hybridized to 50 μg seed or callus total RNA, 3 ng control sense RNA and 10 μg yeast tRNA, or 50 μg yeast tRNA with (negative control) or without (positive control of probe integrity) added RNase at 43.5° C. for 10 h in the hybridization buffer supplied with the kit (RPA III, Ambion). RNase digestion of hybridized probe and sample RNA was performed using an RNase A/T1 mix at a 1:75 dilution in Digestion buffer (Ambion). The samples were electrophoresed at 250 v for 2 h on 5% acrylamide, 8 M urea denaturing gel following a pre-run for 1 h at 350 v. The gel was transferred to filter paper (Whatman 3MM) covered with plastic wrap, and exposed to X-ray film for an appropriate length of time. By utilizing the same PCR primers to generate templates for RPA probes for ZmGOLS2 and 3, two polymorphisms were introduced into the ZmGOLS3 probe relative to the ZmGOLS3 sense RNA. This resulted in the loss of 12 bp from the 5′ end and 9 bp from the 3′ end of the probe reducing its size to 165 bp when it was protected by the ZmGOLS3 RNA. This strategy was necessary to distinguish between ZmGOLS2 and ZmGOLS3 despite 19 RNase sensitive mismatches in the 5′ UTR of the two cDNAs.

Galactinol Synthase Enzyme Assay

Five mature, dehydrated or imbibed maize seeds that had or had not been subjected to various stresses (see germination conditions above), were pulverized in liquid nitrogen in a mortar and pestle and homogenized in ice-cold extraction buffer (50 mM HEPES, 1 mM dithiothreitol [DTT], 50 mM ascorbic acid, 10% [v/v] ethylene glycol, 1 mM MnCl₂, pH 7.5, Liu et al. 1995) for one minute with a Polytron. Following centrifugation (10,000 g, 20 min), the supernatant was assayed directly for galactinol synthase activity in a 25 μl reaction in microfuge tubes. Aliquots (10 μl) of the supernatant were added to assay buffer (50 mM HEPES, 4 mM MnCl₂, 2 mM DTT, 4 μg BSA; Liu et al. 1995) which was then pre-incubated for 15 min at 30° C. prior to the addition of 0.2 μCi uridine-5′-diphosphogalactose-(galactose-6-³H) (Sigma) (16.8 Ci/mmol) and, to one of two duplicate reactions per sample, 20 mM myo-inositol, while to the other water was added to the reaction (Bachmann et al., 1994). Assays proceeded for 16 h and were terminated by the addition of 50 μl 100% ethanol, vortexed, and centrifuged. Un-reacted UDP-Gal was removed by the addition of 100 mg Dowex-1 resin (formate form prepared according to Dawson et al. (1986)) added to each tube and incubated for 20 min prior to the addition of 1 ml water. After vortexing and centrifugation, 500 μl was added to scintillant and the amount of radioactivity determined. Samples to which water rather than seed extract had been added were used to determine the effectiveness of the resin in removing unreacted UDP-galactose from the supernatant. Similar samples without added resin were used to determine assay specific activity for the determination of enzyme activity based on the amount of product formed. Protein concentration in the samples was determined using the Bradford assay (Bradford, 1976) due to its compatibility with the DTT in the extraction buffer.

Sugar Extraction and Analysis

Five mature maize seeds that were unimbibed, had been imbibed for 48 h at 25° C., or had been subjected to salt, heat, cold, or dehydration stress during germination (see above) were pulverized in liquid nitrogen using a mortar and pestle. Additionally, callus tissue that had been growing on media of various compositions were weighed and plunged into liquid nitrogen. The callus was pulverized in liquid nitrogen using a mortar and pestle. Five ml 80% ethanol spiked with 100 mM methyl-glucopyranoside (Sigma) as an internal standard was added to the seed powder or 1 ml per gram fresh weight to the callus powder and ground to produce a slurry. The slurry was transferred to a 15 ml tube, the mortar washed with an additional 2 ml 80% ethanol, and this added to the tube. The suspensions were heated at 80° C. for 20 min, centrifuged at 4000×g, the supernatant collected and diluted to 12 ml with distilled, deionized water. The prolamins precipitating upon addition of water to seed extracts were removed by centrifugation and all supernatants were frozen at −80° C. The extracts were lyopholized to dryness, reconstituted in 1 ml (seed) or 1 ml per gram fresh weight (callus) distilled, deionized water, centrifuged (13,000×g) and 10 μl spotted 2 cm from the bottom of a dry silica gel 60 plate (Sigma) pre-washed in resolving buffer (chloroform:methanol:acetic acid, 70:30:15 v/v; Haer, 1969). The extracts were developed for 4 h in resolving buffer, the plate dried in a fume hood, and then placed back in the TLC tank to develop an additional 4 h. Plates were then dried, sprayed with orcinol ferric chloride (Bial's reagent, Sigma), dried and sugars developed at 100° C. in a convection oven for 15 min.

Characterization of a Drought Resistant Phenotype in Zea Mays rafs 1 Transposon Knock-Outs.

To identify a phenotype associated with a loss of raffinose synthase 1 (RAFS1) activity in Zea mays plants that contain transposon insertions within the RAFS1 gene several different stress experiments were performed on various lines as follows:

Cold Stress Test

A standard cold stress test was performed on the following lines as per the AOSA protocol. Table 1 shows the plant line, generation, and presumed genotype of the 12 different groups.

TABLE 1
Putative genotypes of seeds used for cold stress germination test.

| | | | | 1:2:1 |
|---|---|---|---|---|
| RAFS1 Line | Homo | Hemi | | WT:Hemi:Homo |
| (generation) | Mu | Mu | Homo WT | Mu |
| PV03 124 C07 (F4) | Group 1 | Group 2 | | |
| PV03 47 G04 (F4) | | Group 3 | | Group 4 |
| BT94 133 F04 (F4) | | Group 5 | | Group 6 |
| PV03 124 C07 (F4) | Group 7 | Group 8 | | |
| PV03 47 G04 (F5) | Group 9 | | Group 10 | Group 11 |
| B73 Control | | | Group 12 | |

Homo Mu = transposon present in both chromosomes
Hemi Mu = transposon present in only one chromosome
Homo WT = no transposon present.

For each group, 150 seeds (50 seeds per 3 replications) were used. The results of the cold test are shown in FIG. 11. No significant difference was observed in the ability of the mutants to germinate under cold stress conditions when compared to the wild type (WT) control.

Osmotic Stress

This experiment was conducted in an effort to determine if the rafs1 mutants differ in their ability to germinate under osmotic stress. Seeds from lines PV03 47 G04, PV03 124 C07 (both thought to be homozygous for the transposon), and WT B73 (200 each) were surface sterilized using a protocol adapted from Arabidopsis, which involved treating the seeds with 70% EtOH followed by a solution of bleach and Triton X100. The seeds were then placed in labeled blotter boxes to which first two pieces of autoclaved Kimpac and then one piece of autoclaved Blotter Blue paper had been added. Seeds were arranged on the Blotter Blue paper in a five-by-five grid such that for each of the above three seed groups there were eight boxes. The Kimpac/Blotter Blue paper stack of half of the boxes in each group was tehn wetted with 25 mL of sterile diH₂0 while the other boxes were wetted with 25 mL of filter sterilized −0.5 mPa PEG. The boxes were placed in a 30° C. growth chamber under continuous dark. An image of each box was captured every 12 hours using a Paradigm Imaging System. Each morning after imaging, any excess liquid was removed from the boxes and replaced with 5 mL of new appropriate fluid. No significant difference was found in the ability of these seeds to complete germinatnion under this osmotic stress.

Drought Stress

Following the cold stress test 14 resulting seedlings from each group were selected and planted in individual pots. The pots, six inches in diameter at the top, were labeled with a group number designated in Table 1 and a sequential number 1-14, lined with a single paper towel to prevent loss of soil through the drainage holes, and filled with Metro Mix 510 until total pot weight was 275 g. A single seedling was then placed on the soil in root down posture in the appropriately labeled pot and additional Metro Mix 510 was added until the total pot weight equaled 400 g. The resulting 168 pots were placed on a bench in the green house with continuous light and automatic watering for three days.

Drought stress began on the fourth day. Plants 11, 12, 13, and 14 from each group were watered to soil saturation and allowed to drain excess water for approximately 30 minutes. Approximately 150 mL of Perlite was added to the soil of each pot to reduce evaporative water loss. Each pot was then nested inside another pot into the bottom of which a two-inch square of Parafilm had been placed to seal the pot from evaporative water loss through the drain holes. Each pot was nested in the second pot at the time of weighing. For the next month pot weights were recorded daily at 3 pm. During this period a Li-Cor 6400 was used to measure photosynthesis rates, stomatal conductance, and many other parameters every three days. For these measurements, well-watered plants from groups 1 and 12 served as controls. No cursory correlation was observed between drought stress resilience and genotype at this stage.

Second Drought Stress

Still searching for a phenotype, a second drought stress test was undertaken utilizing the remaining 10 plants from each group. In earlier experiments it was noted an ability of the rafs1 mutants to bear up through repeated cycles of stress and recovery. Also, findings by Anderson and Kohom in their work on Arabidopsis SIP1 raised the possiblity that the drought restraint phenotype could be growth stage dependent and might be seen in plants older than those used in the first drought stress tests (Anderson and Kohom 2001). In order to address both of these possibilities, plants 1-8 were divided into three groups as shown below.

TABLE 2
Treatment groups for second drought stress test.

| Treatment | Individuals | |
|---|---|---|
| group | from each line | Treatment |
| A | 1, 2 | Well watered controls. |
| B | 3, 4, 5, 6 | Pots sealed using the same |
| | | method used in first drought |
| | | stress test. |
| C | 7, 8, 9, 10 | Water withheld for seven |
| | | days, then soil saturated. |
| | | Cycle repeated. |

Initial pot weights for treatment groups B and C were measured and every three days thereafter. An observable difference was noted approximately 8 days later between the Group 1 plants (PV03 124 C07 F4 generation) and Group 12 (B73 WT controls) in treatment B.

There was an obvious difference in the groups with respect to leaf curl. The Group 1 plants still had open leaves, while the curled leaves of the WT B73 plants showed the effect drought stress.

In addition to stress response there appeared to be a difference between the groups based on height and number of green leaves. Table 3 shows the height of each plant measured in centimeters from the soil to the top, outmost blade sheath junction as well as the number of green leaves. In counting green leaves, the leaf had to have no yellowed tissue, regardless of the state of curl. Although there was a noticeable difference between the two groups (FIG. 12), an analysis of variance showed the difference not to be be statiscally significant at this stage. What was significant, however, was the obvious difference in the response of these two groups to drought. Given that larger corn plants use more water than smaller plants, whether Group 1 was statistically larger than Group 12 or not, still true was that Group 1 showed no sign of stress at this stage as evidenced by leaf curl.

TABLE 3
Plant Heights and Numbers of Green Leaves - Treatment B

| | | |
|---|---|---|
| Plant | Height (cm) | Green leaves |

Group 1 Treatment B

| | | |
|---|---|---|
| 1p3 | 42 | 5 |
| 1p4 | 49 | 6 |
| 1p5 | 40 | 4 |
| 1p6 | 48 | 6 |
| Avg. Ht. | 44.75 | 5.25 |

Group 12 Treatment B

| | | |
|---|---|---|
| 12p3 | 39.5 | 4 |
| 12p4 | 45.5 | 4 |
| 12p5 | 34.5 | 4 |
| 12p6 | 38 | 4 |
| Avg. Ht. | 39.375 | 4 |

Similarly, in Treatment C, the Group 1 plants were taller than the Group 12 plants as shown in Table 4 below, although again the differences were not found to be statistically significant at that stage of growth.

TABLE 4
Plant Heights - Treatment C

| | |
|---|---|
| Plant | Height (cm) |

Group 1 Treatment C

| | |
|---|---|
| 1p7 | 45 |
| 1p8 | 45 |
| 1p9 | 46 |
| 1p10 | 42 |
| Avg. Ht. | 44.5 |

Group 12 Treatment C

| | |
|---|---|
| 12p7 | 45.5 |
| 12p8 | 38 |
| 12p9 | 37 |
| 12p10 | 37 |
| Avg. Ht. | 39.375 |

Tassel Production

The Treatment C plants began their third seven-day drought cycle. Two of the Group 1 plants, 7 and 8, received water from a faulty emitter during the first drought cycle. Although much shorter than their well watered counterparts, these two plants had produced visible tassels by the start of their third seven-day drought cycle as had some of the other well watered controls from other groups. By day 4, plant 9 had produced a tassel as had plants 7 and 9 of Group 7. This may indicated tasseling time as another phenotypic marker to look for in the rafs1 mutant lines.

Genotyping of Corn Plants

Concurrent with these experiments, an attempt was made to improve the efficiency of determining whether a given plant was homozygous, hemizygous, or free of the transposon insertion within the RAFS1 gene. Previously, a combination of PCR and Southern blotting were used to type the plants. The PCR reactions were difficult due to the high GC content of corn DNA, and Southern blots are time consuming. Based on others success, we began using the REDExtract-N-Amp Plant PCR Kit produced by Sigma. After a few medications, this kit was an improvement over previous methods.

Evaluation of Pot Weight Data

Using the new technique it was found that some of the supposed genotypes listed in Table 1 above were incorrect. Of particular interest, the Group 10, which was to serve as a control WT for RAFS1 that shared of the peripheral mutation load with the other plants, was found to be segregating for transposon insertion. This meant the F4 plant that was selfed to produce the F5 seed used here was incorrectly typed as being WT when in fact it was most hemizygous for the insert. This discovery proved particularly interesting in light of the pot weight measurements recorded for the Group 10 plants in the first drought stress test. As the data was plotted over time, the four plants sorted themselves into two groups based on weight loss as shown in FIG. 14.

Based on the PCR genotyping of these plants, 10p11 and 10p13 were WT for RAFS1 , while 10p12 and 10p14 were hemizygous for the transposon insert. The correlation of genotype in pot weight loss suggests that the presence of a transposon knocking out one copy of the gene effects the way the plant manages available water. This was an important observation given that the four seeds that produced these plants came from the same ear and therefore shared the same peripheral mutation load. As for how the plants effected this conservation of available moisture, a hint can be gleaned from an examination of stomatal conductance. FIG. 15 shows a plot of conductance rates over time. For the period between day 7 and day 12, the hemizygous plants showed lower conductance than the WT plants.

References

Aalen R B (1999) Peroxiredoxin antioxidants in seed physiology. Seed Sci. Res. 9: 285-295
Adachi T, Izumi H, Yamada T, Tanaka K, Takeuchi S, Nakamura R, Matsuda T (1993) Gene structure and expression of rice seed allergenic proteins belonging to the alpha-amylase/trypsin inhibitor family. Plant Mol. Biol. 21: 239-248
Amuti K S, Pollard C J (1977) Soluble carbohydrates of dry and developing seeds. Phytochem. 16: 529-532.
Anderson C M, Kohom B D (2001) Inactivation of Arabidopsis SIP1 leads to reduced levels of sugars and drought tolerance. J. Plant Physiol. 158: 1215-1219
Armstrong C l, Green C E (1985) Establishment and maintenance of viable, embryogenic maize callus and the involvement of L-proline. Planta 164: 207-214
Ashworth E N, Stirm V E, Volenec J J (1993) Seasonal variations in soluble sugars and starch within woody stems of Cornus sericea L. Tree Physiol. 13: 379-388.
Bachmann M, Matile P, Keller F (1994) Metabolism of the raffinose family oligosaccharides in leaves of Ajuga reptans L. Cold acclimation, translocation, sink to source transition: discovery of chain elongation enzyme. Plant Physiol. 105: 1335-1345
Bachmann M, Keller F (1995) Metabolism of the raffinose family oligosaccharides in leaves of Ajuga reptans L. Inter- and intracellular compartmentation. Plant Physiol. 109: 991-998
Bailly C, Audigier C, Ladonne F, Wagner M H, Coste F, Corbineau F, Côme D (2001) changes in oligosaccharide content and antioxidant enzyme activities in developing bean seeds as related to acquisition of drying tolerance and seed quality. J. Exp. Bot. 52: 701-708
Beebe D U, Turgeon R (1992) Localization of galactinol, raffinose, and stachyose synthesis in Cucurbita pepo leaves. Planta 188: 354-361
Bentsink L, Alonso B C, Vreugdenhil D, Tesnier K, Groot S P C, Koornneef M (2000) Genetic analysis of seed-soluble oligosaccharides in relation to seed storability of Arabidopsis. Plant Physiol. 124: 1595-1604
Bemal-Lugo I, Leopold A C (1992) Changes in soluble carbohydrates during seed storage. Plant Physiol. 98: 1207-1210
Bemal-Lugo I, Leopold A C (1995) Seed stability during storage: raffinose content and seed glassy state. Seed Sci. Res. 5: 75-80
Bemal-Lugo I, Leopold A C (1998) The dynamics of seed mortality. J Exp. Bot. 49: 1455-1461
Bewley J D, Black M (1994) Seeds. Physiology of development and germination. 2nd Edition, Plenum Press, New York, Pgs 126-128 and 137
Black M, Corbineau F, Grzensik M, Guy P, Côme D (1996) Carbohydrate metabolism in the developing and maturing wheat embryo in relation to its desiccation tolerance. J. Exp. Bot. 47: 161-169
Bradford M M (1976) A rapid and sensitive method for the quantitation of microgram quantities of protein utilizing the principle of protein-dye binding. Anal. Biochem. 72: 248-254
Brenac P, Smith M E, Obendorf R L (1997a) Raffinose accumulation in maize embryos in the absence of a fully functional Vp1 gene product. Planta 203: 22-228
Brenac P, Horbowicz M, Downer S M, Dickerman A M, Smith M E, Obendorf R L (1997b) Raffinose accumulation related to desiccation tolerance during maize (Zea mays L.) seed development and maturation. J. Plant Physiol. 150: 481-488
Buckeridge M S, Dietrich S M C (1996) Mobilisation of the raffinose family oligosaccharides and galactomannan in germinating seeds of Sesbania marginalta Benth. (Leguminosae-Faboideae). Plant Sci. 117: 33-43
Buitink J, Claessens M M A E, Hemminga M A, Hoekstra F A (1998) Influence of water content and temperature on molecular mobility and intracellular glasses in seed and pollen. Plant Physiol. 118: 531-541
Buitink J, Hemminga M A, Hoekstra F A (2000) Is there a role for oligosaccharides in seed longevity? An assessment of intracellular glass stability. Plant Physiol. 122: 1217-1224
Carranco R, Almoguera C, Jordano J (1999) An imperfect heat shock element and different upstream sequences are required for the seed-specific expression of a small heat shock protein gene. Plant Physiol. 121: 723-730
Castillo J, Rodrigo I, Marquez J, Zuniga A, Franco L (2000) A pea nuclear protein that is induced by dehydration belongs to the vicilin superfamily. Eur. J. Biochem. 267: 2156-2165
Chen W P, Li P H, Chen T H H (2000) Glycinebetaine increases chilling tolerance and reduces chilling-induced lipid peroxidation in Zea mays L. Plant, Cell and Environment 23: 609-618
Chiou T-J, Bush D R (1998) Sucrose is a signal molecule in assimilate partitioning. Proc. Natl. Acad. Sci. USA. 95: 4784-4788
Chu C C, Wang C C, Sun C S, Hsu C, Yin K C, Chu C Y, Bi F Y (1975). Establishment of an efficient medium for anther culture of rice through comparative experiments on the nitrogen sources. Sci. Sinica. 18: 659-668
Corbineau F, Picard M A, Fougereux J-A, Ladonne F, Côme D (2000) Effects of dehydration conditions on desiccation tolerance of developing pea seeds as related to oligosaccharide content and cell membrane properties. Seed Sci. Res. 10: 329-339
Dawson R M C, Elliott D C, Elliott W H, Jones K M (1986) Data for Biochemical Research. 3^{rd} Edition, Oxford University Press, Oxford, New York, Toronto. Pgs 504-505
Dennehey B K, Petersen W L, Satino C F, Pajeau M, Armstrong C L (1994) Comparison of selective agents for use with the selectable marker gene bar in maize transformation. Plant Cell, Tissue and Organ Culture. 36: 1-7
Denhardt D T (1966) A membrane-filter technique for the detection of complementary DNA. Biochem Biophys Res Comm 23: 641-646
Dirk L M A, van der Krol A R, Vreugdenhil D, Hilhorst H W M, Bewley J D (1999) Galactomannan, soluble sugar, and starch mobilization following germination of fenugreek (Trigonella foenum-graecum L.) seeds. Plant Physiol. Biochem. 37: 41-50
Downie B, Bewley J D (2000) Soluble sugar content of white spruce (Picea glauca) seeds during and after germination. Physiol. Plant. 110: 1-12
Downie B, Gurusinghe S, Dahal P, Nonogaki H, Yim K, Fukanaga K, Alvarado V, Bradford K J (2002) Expression of a galactinol synthase in tomato is stress induced by an ABA-independent mechanism that is differentially responsive to cold in tomato seeds and leaves. Plant Physiol. ______
Duncan D R, Widholm J M (1987) Proline accumulation and its implications in cold tolerance of regenerable maize callus. Plant Physiol. 83: 703-708
Emanuelsson O, Nielsen H, Brunak S, von Heijne G (2000) Predicting subcellular localization of proteins based on their N-terminal amino acid sequence. J. Mol. Biol. 300: 1005-1016
Farrar J, Pollock, C, Gallagher J (2000) Sucrose and the integration of metabolism in vascular plants. Plant Sci. 154: 1-11
Feinburg A P, Vogelstein B (1983) A technique for radiolabelling DNA restriction endonuclease fragments to high specific activity. Anal Biochem 132: 6
Fujiki Y, Ito M, Nishida I, Watanabe A (2000) Multiple signaling pathways in gene expression during sugar starvation. Pharmacological analysis of din gene expression in suspension-cultured cells of Arabidopsis. Plant Physiol. 124: 1139-1147
Fujiki Y, Yoshikawa Y, Sato T, inada N, Ito M, Nishida I, Watanabe A (2001) Dark-inducible genes from Arabidopsis thaliana are associated with leaf senescence and repressed by sugars. Physiol. Plant. 111: 345-352
Gilmour S J, Thomashow M F (1991) Cold acclimation and cold-regulated gene expression in ABA mutants of Arabidopsis thaliana. Plant Mol. Biol. 17: 1233-1240
Gilmour S J, Sebolt A M, Salazar M P, Everard J D, Thomashow M F (2000) Overexpression of the Arabidopsis CBF3 transcriptional activator mimics multiple biochemical changes associated with cold acclimation Plant Physiol. 124: 1854-1865
Golovina E A, Tikhonov A N, Hoekstra F A (1997) An electron paramagnetic resonance spin-probe study of membrane-permeability changes with seed aging. Plant Physiol. 114: 383-389
Grabe D F (1989) measurement of seed moisture. In: Seed moisture. Crop Sci. Soc. Am. Spec. Pub. 14, pp. 69-92
Gurusinghe S, Bradford K J (2001) Galactosyl-sucrose oligosaccharides and potential longevity of primed seeds. Seed Sci. Res. 11: 121-133
Haer, Frederick C (1969) An introduction to chromatography on impregnated glass fiber. Ann Arbor Science Publishers, Ann Arbor, Mich., USA, 173 p
Haritatos E, Medville R, Turgeon R (2000) Minor vein structure and sugar transport in Arabidopsis thaliana. Planta 211: 105-111
Harmer S L, Hogenesch J B, Straume M, Chang H-S, Han B, Zhu T, Wang X, Kreps J A, Kay S A (2000) Orchestrated transcription of key pathways in Arabidopsis by the circadian clock. Science 290: 2110-2113
Henry E W (1976) Determination of inositol in inbred and hybrid corn (Zea mays) seedlings. J. Exp. Bot. 27: 259-262
Heyser W, Heyser R, Eschrich W, Leonard O A, Rautenberg M (1976) The influence of externally applied organic substances on phloem translocation in detached maize leaves. Planta 132: 269-277
Hinesley L E, Pharr D M, Snelling L K, Funderburk S R (1992) Foliar raffinose and sucrose in four conifer species: relationship to seasonal temperature. J. Amer. Soc. Hort. Sci. 117: 852-855
Hitz W D, Carlson T J, Kerr P S, Sebastian S A (2002) Biochemical and molecular characterization of a mutation that confers a decreased raffinosaccharide and phytic acid phenotype on soybean seeds. Plant Physiol. 128: 650-660
Hoekstra F A, Haigh A M, Tetteroo F A A, Van Roekel T (1994) Changes in soluble sugars in relation to desiccation tolerance in cauliflower seeds. Seed Sci. Res. 4: 143-147
Holthaus U, Schmitz K (1991) Distribution and immunolocalization of stachyose synthase in Cucumis melo L. Planta 185: 479-486
Hongtrakul V, Slabaugh M B, Knapp S J (1998) A seed specific delta-12 oleate desaturase gene is duplicated, rearranged, and weakly expressed in high oleic acid sunflower lines. Crop Sci. 38: 1245-1249
Horbowicz M, Obendorf R L (1994) Seed desiccation tolerance and storability: Dependence of flatulence-producing oligosaccharides and cyclitols—review and survey. Seed Sci. Res. 4: 385-405
Ishitani M, Xiong L, Stevenson B, Zhu J-K (1997) Genetic analysis of osmotic and cold stress signal transduction in Arabidopsis: Interactions and convergence of abscisic acid-dependent and abscisic acid-independent pathways. Plant Cell 9: 1935-1949
Keller F, Matile P (1985) The role of the vacuole in storage and mobilization of stachyose in tubers of Stachys sieboldii. J. Plant Physiol. 119: 369-380
Keller F (1992) Transport of stachyose and sucrose by vacuoles of Japanese artichoke (Stachys sieboldii) tubers. Plant Physiol. 98: 442-445
Keller F, Pharr D M (1996) Metabolism of carbohydrates in sinks and sources: Galactosyl-sucrose oligosaccharides. In: Photoassimilate distribution in plants and crops, Source-Sink relationships. (E. Zamski and A A Schaffer, Eds.) Marcel Dekker, Inc., New York. pp. 157-183
Kerr P S, Pearlstein R W, Schweiger B J, Becker-Manley M F, Pierce J W (1997) Nucleotide sequences of galactinol synthase from zucchini and soybean. U.S. Pat. 5,648,210
Koster K L, Leopold A C (1988) Sugars and desiccation tolerance in seeds. Plant Physiol. 88: 829-832
Koster K L (1991) Glass formation and desiccation tolerance in seeds. Plant Physiol. 96: 302-304
Kuo T M, Van Middlesworth J F, Wolf W J (1988) Content of raffinose oligosaccharides and sucrose in various plant seeds. J. Agric. Food Chem. 36: 32-36
Lehle L, Tanner W (1972) Synthesis of raffinose-type sugars. In: Methods in Enzymology. Complex Carbohydrates. (V. Ginsberg, Ed.) Part B, Vol 28. Academic Press, New York, pp 522-529
Leprince O, Hendry G A F, McKersie B D (1993) The mechanisms of desiccation tolerance in developing seeds. Seed Sci. Res. 3: 231-246
Lin T-P, Huang N-H (1994) The relationship between carbohydrate composition of some tree seeds and their longevity. J. Exp. Bot. 45: 1289-1294
Lin X, Kaul S, Rounsley S D, Shea T P, Benito M-I, Town C D, Fujii C Y, Mason T M, Bowman C L, Barnstead M E, Feldblyum T V, Buell C R, Ketchum K A, Lee J J, Ronning C M, Koo H, Moffat K S, Cronin L A, Shen M, VanAken S E, Umayam L, Tallon L J, Gill J E, Adams M D, Carrera A J, Creasy T H, Goodman H M, Somerville C R, Copenhaver G P, Preuss D, Nierman W C, White O, Eisen J A, Salzberg S L, Fraser C M, Venter J C (1999) Sequence and analysis of chromosome 2 of the plant Arabidopsis thaliana. Nature 402: 761-768
Liu J J, Odegard W, de Lumen B O (1995) Galactinol synthase from kidney bean cotyledon and zucchini leaf. Plant Physiol. 109: 505-511
Liu J J, Krenz D C, Galvez A F, de Lumen B O (1998) Galactinol synthase (GS): increased enzyme activity and levels of mRNA due to cold and desiccation. Plant Sci. 134: 11-20
Main E L, Pharr D M, Huber S C, Moreland D E (1983) Control of galactosyl-sugar metabolism in relation to rate of germination. Physiol. Plant. 59: 387-392
Murray M G, Thompson W F (1980) Rapid isolation of high molecular weight DNA. Nucleic Acids Res. 8: 4321-4325
Nichols M B, Bancal M-O, Foley M E, Volenec J J (1993) Non structural carbohydrates in dormant and afterripened wild oat caryopses. Physiol. Plant. 88: 221-228
Nonogaki H, Gee O H, Bradford K J (2000) A germination-specific endo-beta-mannanase gene is expressed in the micropylar endosperm cap of tomato seeds. Plant Physiol. 123: 1235-1246
Ohshima T, Hayashi H, Chino M (1990) Collection and chemical composition of pure phloem sap from Zea mays L. Plant Cell Physiol. 31: 735-737
Ohsumi C, Nozaki J, Kida, T (1998) Characterization and cDNA cloning of raffinose synthase from Cucumis sativus. Plant Physiol. Suppl. Pg 87
Ooms J J J, Léon-Kloosterziel K M, Bartels D, Koornneef M, Karssen C M (1993) Acquisition of desiccation tolerance and longevity in seeds of Arabidopsis thaliana. A comparative study using ABA-insensitive abi3 mutants. Plant Physiol. 102: 1185-1191
Ooms J J J, Wilmer J A, Karssen C M (1994) Carbohydrates are not the sole factor determining desiccation tolerance in seeds of Arabidopsis thaliana. Physiol. Plant. 90: 431-436
Page R D M (1996) TREEVIEW: An application to display phylogenetic trees on personal computers. Computer Applications in the Biosciences 12: 357-358
Pattanagul W, Madore M A (1999) Water deficit effects on raffinose family oligosaccharide metabolism in coleus. Plant Physiol. 121: 987-993
Peterbauer T, Richter A (2001) Biochemistry and physiology of raffinose family oligosaccharides and galactosyl cyclitols in seeds. Seed Sci. Res. 11: 185-197
Peterbauer T, Lahuta L B, Blöchl A, Mucha J, Jones, D A, Hedley C L, Górecki R J, Richter A (2001) Analysis of the raffinose family oligosaccharide pathway in pea seeds with contrasting carbohydrate composition. Plant Physiol. 127: 1764-1772
Pridham J B, Hassid W Z (1965) Biosynthesis of raffinose. Plant Physiol. 40: 984-986
Santarius K A (1973) The protective effect of sugars on chloroplast membranes during temperature and water stress and its relationship to frost, desiccation and heat resistance. Planta 113: 105-114
Santarius K A, Milde H (1977) Sugar compartmentation in frost-hardy and partially dehardened cabbage leaf cells. Planta 136: 163-166
Santarius K A (1996) Freezing of isolated thylakoid membranes in complex media. X. Interactions among various low molecular weight cryoprotectants. Cryobiol. 33: 118-126
Sambrook J, Fritsch E F, Maniatis T (1989) Molecular Cloning. A Laboratory Manual. 2nd edition. Vols I-III, (N Ford, C. Nolan, M. Ferguson eds.) Cold Spring Harbor Laboratory Press
Saravitz D M, Pharr D M, Carter T E (1987) Galactinol synthase activity and soluble sugars in developing seeds of four soybean genotypes. Plant Physiol. 83: 185-189
Sato S, Kotani H, Nakamura Y, Kaneko T, Asamizu E, Fukami M, Miyajima N, Tabata S (1997) Structural analysis of Arabidopsis thaliana chromosome 5. I. Sequence features of the 1.6 Mb regions covered by twenty physically assigned P1 clones. DNA Res. 4: 215-230
Smith P T, Kuo T M, Crawford C G. (1991) Purification and characterization of galactinol synthase from mature zucchini squash leaves. Plant Physiol. 96: 693-698
Sprenger N, Keller F (2000) Allocation of raffinose family oligosaccharides to transport and storage pools in Ajuga reptans: the roles of two distinct galactinol synthases. Plant J. 21: 249-258
Sun W Q, Leopold A C (1993) The glassy state and accelerated aging of soybeans. Physiol. Plant. 89: 767-774
Sun W Q, Irving T C, Leopold A C (1994) The role of sugar, vitrification and membrane phase transition in seed desiccation tolerance. Physiol. Plant. 90: 621-628
Swofford D L (1998) PAUP*. Phylogenetic Analysis Using Parsimony (*and Other Methods). Version 4. Sinauer Associates, Sunderland, Mass., USA
Taji T, Ohsumi C, Luchi S, Seki M, Kasuga M, Kobayashi M, Yamaguchi-Shinozaki K, Shinozaki K (2002) Important roles of drought- and cold-inducible genes for galactinol synthase in stress tolerance in Arabidopsis thaliana. Plant J. 29: 417-426
Takahashi R, Joshee N, Kitagawa Y (1994) Induction of chilling resistance by water stress, and cDNA sequence analysis and expression of water stress-regulated genes in rice. Plant Mol. Biol. 26: 339-352
Thompson J D, Higgins D G, Gibson T J (1994) CLUSTAL W: improving the sensitivity of progressive multiple sequence alignment through sequence weighting, position-specific gap penalties and weight matrix choice. Nucleic Acids Res. 22: 4673-4680
Vain P, Mcmullen M D, Finer J J (1993) Osmotic treatment enhances particle bombardment-mediated transient and stable transformation of maize. Plant Cell Rep. 12: 84-88
van Bel A J E, Hendriks J H M, Boon E J M C, Gamalei Y V, van de Merwe A P (1996) Different ratios of sucrose/raffinose-induced membrane depolarizations in the mesophyll of species with symplasmic (Catharanthus roseus, Ocimum basilicum) or apoplasmic (Impatiens walleriana, Vicia faba) minor-vein configurations. Planta 199: 185-192
Wan C-Y, Wilkins T A (1994) A modified hot borate method significantly enhances the yield of high quality RNA from cotton (Gossypium hirsutum L.). Anal. Biochem. 223: 7-12
Wang G, Du T, Zhang H, Xie Y, Dai J, Mi J, Li T, Tian Y, Qiao L, Mang K (1995) Transfer of Bt toxin-protein gene into maize by high-velocity microprojectile bombardments and regeneration of transgenic plants. Science in China (B) 39: 817-824
Weiner H, Blechschmidt-Schneider S, Mohme H, Eschrich W, Heldt H W (1991) Phloem transport of amino acids. Comparison of amino acid contents of maize leaves and of the sieve tube exudate. Plant Physiol. Biochem. 29: 19-23
Wiemken V, Ineichen K (1993) Effect of temperature and photoperiod on the raffinose content of spruce roots. Planta 190: 387-392
Wohlfarth T, Braun H, Kirik V, Koelle K, Czihal A, Tewes A, Luerssen H, Misera S, Shutov A, Baeumlein H (1998) Regulation and evolution of seed globulin genes. J Plant Physiol. 152: 600-606
Wu Y, Meeley R B, Cosgrove D J (2001) Analysis and expression of the alpha-expansin and beta-expansin gene families in maize. Plant Physiol. 126: 222-232
Xiao L, Koster K L (2001) Desiccation tolerance of protoplasts isolated from pea embryos. J. Exp. Bot. 364: 2105-2114

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims. Thus, many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims.

1. An isolated nucleic acid molecule, said molecule encoding a heterologous stress-responsive protein wherein said nucleic acid molecule comprises a nucleotide sequence selected from the group consisting of:
(a) a nucleotide sequence comprising the sequence set forth in SEQ ID NOS: 1, 3, 5,7,9,or 11,
(b) a nucleotide sequence comprising at least 20 contiguous nucleotides of the sequence of SEQ ID NOS: 1, 3, 5, 7, 9, or 11, wherein said nucleotide sequence encodes a protein with stress-responsive activity;
(c) a nucleotide sequence having at least 70% sequence identity to the sequence of SEQ ID NOS: 1, 3, 5, 7, 9, or 11, wherein said nucleotide sequence encodes a protein with stress-responsive activity.

2. A vector comprising the nucleic acid molecule of claim 1.

3. A plant cell having stably incorporated in its genome the nucleic acid molecule of claim 1.

4. The plant cell of claim 3, wherein said plant cell is from a dicot plant.

5. The plant cell of claim 4, wherein said dicot plant is soybean.

6. The plant cell of claim 3 wherein said plant cell is a root cell.

7. A nucleic acid molecule that drives expression of an operably linked nucleic acid sequence in a plant cell, wherein said nucleic acid molecule which drives expression comprises a nucleotide sequence selected from the group consisting of:
(a) a maize nucleotide sequence natively associated with a GOLS or RAFS encoding nucleotide sequence,
(b) a nucleotide sequence natively associated with SEQ ID NOS: 1, 3, 5, 7, 9, or 11;

8. A vector comprising the nucleic acid molecule of claim 7.

9. A plant cell having stably incorporated in its genome the nucleic acid molecule of claim 7.

10. The plant cell of claim 9, wherein the plant cell is from a dicot plant.

11. The plant cell of claim 10, wherein the dicot plant is soybean.

12. The plant cell of claim 9 wherein said cell is a root cell.

13. A method for conferring or improving stress resistance in a plant, said method comprising:
transforming said plant with a nucleic acid molecule comprising a heterologous sequence operably linked to a promoter that induces transcription of said heterologous sequence in a plant cell in response to a stress stimulus; and
regenerating stably transformed plants, wherein said heterologous sequence comprises a nucleotide sequence selected from the group consisting of:
(a) a nucleotide sequence comprising the sequence set forth in SEQ ID NOS: 1, 3,5,7,9,or 11;
(b) a nucleotide sequence comprising at least 50 contiguous nucleotides of the sequence of SEQ ID NOS: 1, 3, 5, 7, 9, or 11, wherein said nucleotide sequence encodes a protein with stress responsive activity; and
(c) a nucleotide sequence having at least 70% sequence identity to the sequence of SEQ ID NOS: 1, 3, 5, 7, 9, or 11.

14. The method of 31, wherein said plant is a dicot.

15. The method of 32, wherein said dicot is soybean.

16. An isolated polypeptide having stress regulatory-like activity and selected from the group consisting of:
(a) a polypeptide comprising the amino acid sequence set forth in SEQ ID NOS:2, 4, 6, 8, 10, or 12;
(b) a polypeptide encoded by a nucleotide sequence comprising the sequence set forth in SEQ ID NOS: 1, 3, 5, 7, 9, or 11;
(c) a polypeptide comprising an amino acid sequence encoded by a nucleotide sequence deposited as Deposit No. PTA-______;
(d) a polypeptide encoded by a nucleotide sequence that has at least 70% sequence identity to the sequence set forth in SEQ ID NOS: 1, 3, 5, 7, 9, or 11.
(e) a polypeptide comprising an amino acid sequence having at least 50% sequence identity to the sequence set forth in SEQ ID NO:2, 4, 6, 8, 10 or 12; and
(f) a polypeptide comprising an amino acid sequence of at least 30 consecutive amino acids of any of (a) through (e).

17. An isolated nucleic acid molecule that encodes a polypeptide having GOLS or RAFS-like activity, said nucleic acid molecule being selected from the group consisting of:
(a) a nucleic acid molecule comprising the sequence set forth in SEQ ID NOS: 1, 3, 5, 7, 9, or 11;
(b) a nucleic acid molecule comprising a sequence encoding the amino acid sequence set forth in SEQ ID NOS: 2, 4, 6, 8, 10 or 12;
(c) a nucleic acid molecule comprising a sequence deposited as Deposit No. PTA-______;
(d) a nucleic acid molecule comprising an antisense sequence corresponding to a sequence of a), b), or c);
(e) a nucleic acid molecule comprising a sequence having at least 70% sequence identity to the nucleotide sequence set forth in SEQ ID NOS: 1, 3, 6, 8, 10, 12, 14, or 16; and
(f) a nucleic acid molecule comprising a sequence of at least 50 consecutive nucleic acids of any of (a) through (e).

18. A nucleotide construct comprising:
a nucleic acid molecule of claim 35, wherein said nucleic acid molecule is operably linked to a promoter that drives expression in a host cell.

19. A method for modulating a peptide comprising:
transforming a host cell with the nucleic acid molecule of claim 35.

20. A cell having stably incorporated into its genome at least one nucleotide construct comprising:
a nucleic acid molecule operably linked to a heterologous promoter that drives expression in said cell, wherein said nucleic acid molecule encodes a polypeptide having RAFS or GOLS-like activity and is selected from the group consisting of:
(a) a nucleic acid molecule comprising the sequence set forth in SEQ ID NOS: 1, 3, 5, 7, 9,or 11;
(b) a nucleic acid molecule comprising a sequence encoding the amino acid sequence set forth in SEQ ID NOS: 2, 4, 6, 8, 10, or 12;
(c) a nucleic acid molecule comprising a sequence deposited as Deposit No. PTA-______;
(d) a nucleic acid molecule comprising an antisense sequence corresponding to a sequence of a), b), or c);
(e) a nucleic acid molecule comprising a sequence having at least 70% sequence identity to the nucleotide sequence set forth in SEQ ID NOS: 1, 3, 5, 7, 9, or 11; and
(f) a nucleic acid molecule comprising a sequence of at least 50 consecutive nucleic acids of any of (a) through (e).

21. The cell of claim 38, wherein said cell is a plant cell.

22. A transformed plant having stably incorporated into its genome at least one nucleotide construct comprising:
a nucleic acid molecule operably linked to a heterologous promoter that drives expression in a plant cell, wherein said nucleic acid molecule encodes a polypeptide having GOLS or RAFS-like activity and is selected from the group consisting of:
(a) a nucleic acid molecule comprising the sequence set forth in SEQ ID NOS: 1, 3, 5, 7, 9, or 11
(b) a nucleic acid molecule comprising a sequence encoding the amino acid sequence set forth in SEQ ID NOS: 2, 4, 6, 8, 10, and 12;
(c) a nucleic acid molecule comprising a sequence deposited as Deposit No. PTA-______;
(d) a nucleic acid molecule comprising an antisense sequence corresponding to a sequence of a), b), or c);
(e) a nucleic acid molecule comprising a sequence having at least 70% sequence identity to the nucleotide sequence set forth in SEQ ID NOS: 1, 3, 5, 7, 9, or 11; and
(f) a nucleic acid molecule comprising a sequence of at least 50 consecutive nucleic acids of any of (a) through (e).

23. The plant of claim 40, wherein said promoter is a constitutive promoter.

24. The plant of claim 40, wherein said promoter is a tissue-preferred promoter.

25. The plant of claim 40, wherein said promoter is an inducible promoter.

26. The plant of claim 40, wherein said plant is a monocot.

27. The plant of claim 40, wherein said monocot is maize, wheat, rice, barley, sorghum, or rye.

28. The plant of claim 40, wherein said plant is a dicot.

29. Transformed seed of the plant of claim 40.

30. A method for conferring or improving stress resistance of a plant, said method comprising:
stably introducing into the genome of a plant, at least one nucleotide construct comprising a nucleic acid molecule operably linked to a heterologous promoter that drives expression in a plant cell, wherein said nucleic acid molecule encodes a polypeptide having GOLS or RAFS-like activity and is selected from the group consisting of:
(a) a nucleic acid molecule comprising the sequence set forth in SEQ ID NOS: 1, 3, 5, 7, 9 or 11;
(b) a nucleic acid molecule comprising a sequence encoding the amino acid sequence set forth in SEQ ID NOS2, 4, 6, 8, 10 or 12;
(c) a nucleic acid molecule comprising a sequence deposited as Deposit No. PTA-______;
(d) a nucleic acid molecule comprising an antisense sequence corresponding to a sequence of a), b), or c);
(e) a nucleic acid molecule comprising a sequence having at least 70% sequence identity to the nucleotide sequence set forth in SEQ ID NOS: 1, 3, 5, 7, 9, or 11; and
(f) a nucleic acid molecule comprising a sequence of at least 50 consecutive nucleic acids of any of (a) through (e).

31. An isolated nucleic acid molecule that encodes a polypeptide having GOLS-like activity, said nucleic acid molecule being selected from the group consisting of:
(a) a nucleic acid molecule comprising the sequence set forth in SEQ ID NOS: 1, 3, 5, 7, 9, or 11;
(b) a nucleic acid molecule comprising a sequence encoding the amino acid sequence set forth in SEQ ID NOS: 2, 4, 6, 8, 10 or 12;
(c) a nucleic acid molecule comprising a sequence deposited as Deposit No. PTA-______;
(d) a nucleic acid molecule comprising an antisense sequence corresponding to a sequence of a), b), or c);
(e) a nucleic acid molecule comprising a sequence having at least 70% sequence identity to the nucleotide sequence set forth in SEQ ID NOS: 1, 3, 6, 8, 10, 12, 14, or 16; and
(f) a nucleic acid molecule comprising a sequence of at least 50 consecutive nucleic acids of any of (a) through (e).
(g)

32. An isolated nucleic acid molecule that encodes a polypeptide having RAFS-like activity, said nucleic acid molecule being selected from the group consisting of:
(a) a nucleic acid molecule comprising the sequence set forth in SEQ ID NOS: 1,3,5,7,9,or 11;
(b) a nucleic acid molecule comprising a sequence encoding the amino acid sequence set forth in SEQ ID NOS: 2, 4, 6, 8, 10 or 12;
(c) a nucleic acid molecule comprising a sequence deposited as Deposit No. PTA-______;
(d) a nucleic acid molecule comprising an antisense sequence corresponding to a sequence of a), b), or c);
(e) a nucleic acid molecule comprising a sequence having at least 70% sequence identity to the nucleotide sequence set forth in SEQ ID NOS: 1, 3, 6, 8, 10, 12, 14, or 16; and
(f) a nucleic acid molecule comprising a sequence of at least 50 consecutive nucleic acids of any of (a) through (e).

33. A method for directing raffinose production in a plant, said method comprising:
stably introducing into the genome of a plant, at least one nucleotide construct comprising a nucleic acid molecule operably linked to a heterologous promoter that drives expression in a plant cell, wherein said nucleic acid molecule encodes a polypeptide having GOLS or RAFS-like activity and is selected from the group consisting of:
(a) a nucleic acid molecule comprising the sequence set forth in SEQ ID NOS: 1,3,5,7,9 or 11;
(b) a nucleic acid molecule comprising a sequence encoding the amino acid sequence set forth in SEQ ID NOS2, 4, 6, 8, 10 or 12;
(c) a nucleic acid molecule comprising a sequence deposited as Deposit No. PTA-______;
(d) a nucleic acid molecule comprising an antisense sequence corresponding to a sequence of a), b), or c);
(e) a nucleic acid molecule comprising a sequence having at least 70% sequence identity to the nucleotide sequence set forth in SEQ ID NOS: 1, 3, 5, 7, 9, or 11; and
(f) a nucleic acid molecule comprising a sequence of at least 50 consecutive nucleic acids of any of (a) through (e).
